(19) United States
(12) Patent Application Publication
(10) Pub. No.: US 2024/0058328 A1
(43) Pub. Date: Feb. 22, 2024

(54) MASITINIB FOR THE TREATMENT OF ALZHEIMER'S DISEASE

(71) Applicant: AB SCIENCE, Paris (FR)

(72) Inventors: Alain MOUSSY, Paris (FR); Colin MANSFIELD, Saint-Contest (FR)

(73) Assignee: AB SCIENCE, Paris (FR)

(21) Appl. No.: 18/256,721

(22) PCT Filed: Dec. 16, 2021
(86) PCT No.: PCT/EP2021/086308

§ 371 Date: Jun. 9, 2023

(30) Foreign Application Priority Data
Dec. 16, 2020 (EP) 20306589.1

(51) Int. Cl. A61K 31/496; A61K 31/44; A61K 31/27; A61K 31/55; A61K 31/13; A61P 25/28

(52) U.S. Cl. CPC A61K 31/496; A61K 31/44; A61K 31/27; A61K 31/55; A61K 31/13; A61P 25/28

(57) ABSTRACT

A 2-aminoarylthiazole derivative, in particular masitinib, or a pharmaceutically acceptable salt or solvate thereof, for use in the treatment of less severe and/or early-stage Alzheimer's disease in a patient in need thereof. In particular, masitinib, or a pharmaceutically acceptable salt or solvate thereof, for use in the treatment of Alzheimer's disease in a patient having a baseline ADCS-ADL (Alzheimer's Disease Cooperative Study—Activities of Daily Living) score equal to or greater, preferably greater than 32, having a baseline MMSE (Mini-Mental State Examination) score equal to or greater than 13, having a time from diagnosis to treatment initiation with masitinib, or a pharmaceutically acceptable salt or solvate thereof, equal to or less than 5 years, and/or having a baseline ADAS-Cog (Alzheimer's Disease Assessment Scale—Cognitive Subscale) score equal to or lower than 40.

### FIELD OF INVENTION

The present invention relates to the treatment of Alzheimer's disease in a patient in need thereof, in particular to the treatment of less severe Alzheimer's disease and/or early-stage Alzheimer's disease or Alzheimer's dementia.

### BACKGROUND OF INVENTION

Alzheimer's disease (AD) is the most common cause of dementia among older adults, notably in western countries where it is estimated to account for about 60%-70% of all dementia cases. Dementia describes a syndrome characterized by dysmnesia (i.e., memory disorder), intellectual deterioration, personality changes and behavioral abnormalities. These symptoms ultimately result in social and occupational decline.

Alzheimer's disease is an irreversible, neurodegenerative disorder leading to disabling impairment of memory and cognitive skills. Alzheimer's disease starts with mild cognitive problems, such as memory loss, ultimately progressing to the stage where independent living is no longer possible. The principal risk factor for developing Alzheimer's disease is age, with the likelihood of developing the disease doubling about every five years after 65 years of age. About one-third of all individual of 85 years of age and older may have Alzheimer's disease. A family history also increases the risk of developing the disease, which may be due to genetics or environmental factors.

The two histopathological features of Alzheimer's disease observed in the brain are amyloid plaques (also referred to as neuritic plaques or senile plaques) which develop between neurons, and neurofibrillary tangles which develop within neurons. Amyloid plaques result from the aggregation of β-amyloid peptide that is generated by the cleavage of the amyloid precursor protein. Neurofibrillary tangles predominantly result from the abnormal accumulation of the phosphorylated protein tau (τ), which links together to form filaments. Deposition of amyloid plaques and formation of neurofibrillary tangles are thought to be intricately related to the cause, development and course of the disease.

The meta-analysis of epidemiologic studies indicates that between 5 and 10 million individuals suffer from Alzheimer's disease in the USA and Europe. Alzheimer's disease is already the sixth leading cause of all deaths in USA and the fifth leading cause among Americans over 65 years of age. It is estimated that there are over three million individuals with dementia in the European Union, and of these about 70% suffer from Alzheimer's disease. It is predicted that global prevalence of Alzheimer's disease will quadruple by 2050 to more than 100 million, at which time 1 in 85 individuals worldwide will be living with the disease. More than 40 percent of those cases will be in late-stage Alzheimer's requiring a high level of attention equivalent to nursing home care.

Currently, the approved treatments for Alzheimer's disease consist of two types of medication: cholinesterase inhibitors (such as donepezil, rivastigmine, and galantamine) and a NMDA (N-methyl-D-aspartate) receptor antagonist (memantine). Cholinesterase inhibitors increase cholinergic transmission by inhibiting acetylcholinesterase at the synaptic cleft. Memantine is a voltage-dependent, moderate-affinity uncompetitive NMDA-receptor antagonist. It modulates the effects of pathologically elevated tonic levels of glutamate that may lead to neuronal dysfunction. The currently approved treatments have shown an efficacy on some cognitive and non-cognitive symptoms of Alzheimer disease. However, their efficacy is limited and may decrease with time.

Therefore, there is still a need for effective treatments for Alzheimer's disease. In particular, there is still a need for effective treatments for less severe Alzheimer's disease and/or early-stage Alzheimer's disease or Alzheimer's dementia.

The present invention thus relates to a 2-aminoarylthiazole derivative, in particular masitinib, or a pharmaceutically acceptable salt or solvate thereof, for use in the treatment of Alzheimer's disease in a patient in need thereof, in particular in a patient suffering from less severe Alzheimer's disease and/or early-stage Alzheimer's disease or Alzheimer's dementia as defined herein.

### SUMMARY

The present invention relates to masitinib, or a pharmaceutically acceptable salt or solvate thereof, for use in the treatment of Alzheimer's disease in a patient in need thereof, wherein before treatment initiation with masitinib, or a pharmaceutically acceptable salt or solvate thereof, the patient suffering from Alzheimer's disease has an Alzheimer's Disease Cooperative Study—Activities of Daily Living (ADCS-ADL) score equal to or greater, preferably greater, than 32, and/or has a Mini-Mental State Examination (MMSE) score equal to or greater than 13, and/or has a time from diagnosis to treatment initiation with masitinib, or a pharmaceutically acceptable salt or solvate thereof, equal to or less than 5 years, and/or has an Alzheimer's Disease Assessment Scale—Cognitive Subscale (ADAS-Cog) score equal to or lower than 40.

In one embodiment, the patient suffering from Alzheimer's disease has an ADCS-ADL score equal to or greater, preferably greater, than 32, 35, 38, 39, 41, 47, 50, or 55, before treatment initiation with masitinib, or a pharmaceutically acceptable salt or solvate thereof. In one embodiment, the patient suffering from Alzheimer's disease has an ADCS-ADL score equal to or greater than 38 or 50 before treatment initiation with masitinib, or a pharmaceutically acceptable salt or solvate thereof. In one embodiment, the patient suffering from Alzheimer's disease has an ADCS-ADL score greater than 39 or 55 before treatment initiation with masitinib, or a pharmaceutically acceptable salt or solvate thereof.

In one embodiment, the patient suffering from Alzheimer's disease has a MMSE score equal to or greater than 13, 14, 15, 16, or 17, preferably equal to or greater than 14, before treatment initiation with masitinib, or a pharmaceutically acceptable salt or solvate thereof. In one embodiment, the patient suffering from Alzheimer's disease has a MMSE score ranging from 21 to 25 before treatment initiation with masitinib, or a pharmaceutically acceptable salt or solvate thereof.

In one embodiment, the patient suffering from Alzheimer's disease has a time from diagnosis to treatment initiation with masitinib, or a pharmaceutically acceptable salt or solvate thereof, equal to or less than 5 years, 4 years, 3 years, or 2 years, preferably equal to or less than 3 years.

In one embodiment, the patient suffering from Alzheimer's disease has an ADAS-Cog score equal to or lower than 40, 35, 32, or 25 before treatment initiation with masitinib, or a pharmaceutically acceptable salt or solvate thereof. In one embodiment, the patient suffering from Alzheimer's disease has an ADAS-Cog score equal to or lower than 35 before treatment initiation with masitinib, or a pharmaceutically acceptable salt or solvate thereof.

In one embodiment, the pharmaceutically acceptable salt of masitinib is masitinib mesilate.

In one embodiment, masitinib, or a pharmaceutically acceptable salt or solvate thereof, is for administration at a dose ranging from about 1 to about 12 mg/kg/day (mg per kilo body weight per day), preferably at a dose of about 3 mg/kg/day, 4.5 mg/kg/day, or 6 mg/kg/day. In one embodiment, masitinib, or a pharmaceutically acceptable salt or solvate thereof, is for administration at an initial dose of about 4.5 mg/kg/day during at least 12 weeks, and then at a dose of about 6 mg/kg/day thereafter, with each dose escalation being subjected to toxicity controls. In one embodiment, masitinib, or a pharmaceutically acceptable salt or solvate thereof, is for oral administration. In one embodiment, masitinib, or a pharmaceutically acceptable salt or solvate thereof, is for administration in two daily intakes.

In one embodiment, masitinib, or a pharmaceutically acceptable salt or solvate thereof, is for administration with at least one further pharmaceutically active agent. In one embodiment, the at least one further pharmaceutically active agent is selected from the group consisting of donepezil, rivastigmine, galantamine, and memantine.

### Definitions

In the present invention, the following terms have the following meanings:

“About” preceding a FIGURE encompasses plus or minus 10%, or less, of the value of said FIGURE. It is to be understood that the value to which the term “about” refers is itself also specifically, and preferably, disclosed.

“Baseline” as used herein refers to the time preceding the start of the treatment with the 2-aminoarylthiazole derivative, in particular masitinib, or a pharmaceutically acceptable salt or solvate thereof, as described herein. In one embodiment, “baseline” corresponds to the state before treatment initiation with the 2-aminoarylthiazole derivative, in particular masitinib, or a pharmaceutically acceptable salt or solvate thereof, as described herein. For example, in one embodiment, a baseline score is a score before treatment initiation with the 2-aminoarylthiazole derivative, in particular masitinib, or a pharmaceutically acceptable salt or solvate thereof, as described herein. In one embodiment, “baseline” corresponds to the state at the time of treatment initiation with the 2-aminoarylthiazole derivative, in particular masitinib, or a pharmaceutically acceptable salt or solvate thereof, as described herein. For example, in one embodiment, a baseline score is a score at the time of treatment initiation with the 2-aminoarylthiazole derivative, in particular masitinib, or a pharmaceutically acceptable salt or solvate thereof, as described herein.

“Pharmaceutically acceptable excipient” or “pharmaceutically acceptable carrier” refers to an excipient or carrier that does not produce an adverse, allergic or other untoward reaction when administered to a patient. It includes any and all solvents, such as, for example, dispersion media, coatings, antibacterial and antifungal agents, isotonic, and absorption delaying agents. A pharmaceutically acceptable excipient or carrier refers to a non-toxic solid, semi-solid or liquid filler, diluent, encapsulating material, or formulation auxiliary of any type. For human administration, preparations should meet sterility, pyrogenicity, general safety and purity standards as required by the regulatory offices such as the FDA (U.S. Food and Drug Administration) or EMA (European Medicines Agency).

“Patient” refers to a human subject suffering from Alzheimer's disease. In one embodiment, the patient is awaiting the receipt of, or is receiving medical care or was/is/will be the object of a medical procedure, or is monitored for the development of Alzheimer's disease.

“AD patient” as used herein refers to a patient suffering from Alzheimer's disease (AD).

“Progression” in “Alzheimer's disease progression (AD progression)” refers to the gradual worsening over time of symptoms, in particular to the gradual worsening of memory impairment and/or cognitive impairment, during the disease course. AD progression may for example be assessed with the Alzheimer's Disease Assessment Scale—Cognitive Subscale (ADAS-Cog), the Alzheimer's Disease Cooperative Study—Activities of Daily Living (ADCS-ADL) scale, and/or the Mini-Mental State Examination (MMSE).

“Progression of score”, such as progression of ADAS-Cog score, progression of ADCS-ADL score, or progression of MMSE score, as used herein refers to the change over time in the score used to assess a patient suffering from Alzheimer's disease (such as the ADAS-Cog score, the ADCS-ADL score, or the MMSE score). As the disease progresses, the score may increase (i.e., ADAS-Cog score) or decrease (i.e., ADCS-ADL score, or MMSE score). The progression of score may be used to assess the efficacy of a treatment. In one embodiment, the progression of score thus corresponds to the difference between the score at baseline (i.e., before treatment initiation) and the score once treatment has been initiated (measured at a specific timepoint following treatment initiation). The absence of difference in the score over time indicates an absence of disease progression and a stable state of the AD patient. For the ADAS-Cog score, a small increase (or a positive difference between a given timepoint and baseline) indicates a limited progression of the disease. Thus, the smaller the increase in the ADAS-Cog score (i.e., the smaller the positive difference), the more stable the AD patient. Accordingly, a smaller increase in the ADAS-Cog score, as compared to the progression of ADAS-Cog score over an identical time period before treatment, indicates a slowing-down of the progression of the disease. Conversely, a decrease in the ADAS-Cog score (or a negative difference between a given timepoint and baseline) indicates an absence of disease progression and an improvement of the AD patient. For the ADCS-ADL score and/or MMSE score, a small decrease (or a negative difference between a given timepoint and baseline) indicates a limited progression of the disease. Thus, the smaller the decrease in the ADCS-ADL score and/or MMSE score (i.e., the smaller the negative difference), the more stable the AD patient. Accordingly, a smaller decrease in the ADCS-ADL score and/or MMSE score, as compared to the progression of ADCS-ADL score and/or MMSE score over an identical time period before treatment, indicates a slowing-down of the progression of the disease. Conversely, an increase in the ADCS-ADL score and/or MMSE score (or a positive difference between a given timepoint and baseline) indicates an absence of disease progression and an improvement of the AD patient.

“Therapeutically effective amount” or “therapeutically effective dose” refers to the amount or concentration of a 2-aminoarylthiazole derivative as defined herein, in particular masitinib, or a pharmaceutically acceptable salt or solvate thereof, that is aimed at, without causing significant negative or adverse side effects to the patient in need of treatment, preventing, reducing, or slowing down (lessening) memory impairment and/or cognitive impairment in the AD patient. In one embodiment, the therapeutically effective amount or therapeutically effective dose is aimed at, without causing significant negative or adverse side effects to the patient in need of treatment, bringing about at least one of the following:
slowing-down of the progression of Alzheimer's disease corresponding to a slowing-down of the increase of the ADAS-Cog score over time, a stability of the ADAS-Cog score over time, or a decrease of the ADAS-Cog score over time;
slowing-down of the progression of Alzheimer's disease corresponding to a slowing-down of the decrease of the ADCS-ADL score over time, a stability of the ADCS-ADL score over time, or an increase of the ADCS-ADL score over time; and/or
slowing-down of the progression of Alzheimer's disease corresponding to a slowing-down of the decrease of the MMSE score over time, a stability of the MMSE score over time, or an increase of the MMSE score over time.

“Treating” or “Treatment” refers to a therapeutic treatment, to a prophylactic (or preventative) treatment, or to both a therapeutic treatment and a prophylactic (or preventative) treatment, wherein the object is to prevent, reduce, or slow down (lessen) one or more of the symptoms or manifestations of Alzheimer's disease in the patient in need of treatment. In one embodiment, the object of the treatment according to the present application is to prevent, reduce, or slow down (lessen) memory impairment and/or cognitive impairment in the AD patient in need of treatment. In one embodiment, the object of the treatment according to the present application is to bring about at least one of the following:
slowing-down of the progression of Alzheimer's disease corresponding to a slowing-down of the increase of the ADAS-Cog score over time, a stability of the ADAS-Cog score over time, or a decrease of the ADAS-Cog score over time;
slowing-down of the progression of Alzheimer's disease corresponding to a slowing-down of the decrease of the ADCS-ADL score over time, a stability of the ADCS-ADL score over time, or an increase of the ADCS-ADL score over time; and/or
slowing-down of the progression of Alzheimer's disease corresponding to a slowing-down of the decrease of the MMSE score over time, a stability of the MMSE score over time, or an increase of the MMSE score over time.

“Time from diagnosis to treatment initiation” as used herein refers to the length of time between date of first clinically definite diagnosis of Alzheimer's disease (expressed as day/month/year, month/year, or year) and date of treatment initiation with the 2-aminoarylthiazole derivative as described herein, in particular masitinib, or a pharmaceutically acceptable salt or solvate thereof (expressed as day/month/year, month/year, or year). In one embodiment, diagnosis of Alzheimer's disease is based on the DSM-IV criteria (Diagnostic and Statistical Manual of Mental Disorders, 4^{th} Edition) and/or to the NINCDS-ADRDA criteria (National Institute of Neurological and Communicative Disorders and Stroke, and Alzheimer's Disease and Related Disorders Association). In one embodiment, if the date is expressed as month/year (i.e., if information regarding the day is missing), the day is considered to be the last day of the month. In one embodiment, if the date is expressed as a year (i.e., if information regarding the day and the month are missing), the month is considered to be June and the day is considered to be the last day of the month, i.e., the last day of June.

### DETAILED DESCRIPTION

The present invention relates to a 2-aminoarylthiazole derivative as described herein, in particular masitinib, or a pharmaceutically acceptable salt or solvate thereof, for use in the treatment of Alzheimer's disease in a patient in need thereof as defined hereinafter. According to one embodiment, the present invention relates to a 2-aminoarylthiazole derivative as described herein, in particular masitinib, or a pharmaceutically acceptable salt or solvate thereof, for use in the treatment of less severe and/or early-stage Alzheimer's disease in a patient in need thereof, in particular in a patient in need thereof as defined hereinafter. According to one embodiment, the present invention relates to a 2-aminoarylthiazole derivative as described herein, in particular masitinib, or a pharmaceutically acceptable salt or solvate thereof, for use in the treatment of less severe Alzheimer's disease in a patient in need thereof, in particular in a patient in need thereof as defined hereinafter. According to one embodiment, the present invention relates to a 2-aminoarylthiazole derivative as described herein, in particular masitinib, or a pharmaceutically acceptable salt or solvate thereof, for use in the treatment of early-stage Alzheimer's disease or early-stage Alzheimer's dementia in a patient in need thereof, in particular in a patient in need thereof as defined hereinafter.

In particular, in one embodiment, the present invention relates to a 2-aminoarylthiazole derivative as described herein, in particular masitinib, or a pharmaceutically acceptable salt or solvate thereof, for use in the treatment of Alzheimer's disease in a patient having an Alzheimer's Disease Assessment Scale—Cognitive Subscale (ADAS-Cog) score equal to or lower than 60 before treatment initiation, having an Alzheimer's Disease Cooperative Study—Activities of Daily Living (ADCS-ADL) score equal to or greater, preferably greater, than 20 before treatment initiation, having a Mini-Mental State Examination (MMSE) score equal to or greater than 12 before treatment initiation, and/or having a time from diagnosis to treatment initiation with masitinib, or a pharmaceutically acceptable salt or solvate thereof, equal to or less than 10 years.

As used herein, “before treatment initiation” means before treatment initiation with a 2-aminoarylthiazole derivative as described herein, in particular masitinib, or a pharmaceutically acceptable salt or solvate thereof. In other words, “before treatment initiation” means at baseline. Thus, in one embodiment, a score (such as an ADAS-Cog score, an ADCS-ADL score, or a MMSE score) before treatment initiation is a baseline score (such as a baseline ADAS-Cog score, a baseline ADCS-ADL score, or a baseline MMSE score).

According to one embodiment, the present invention relates to a 2-aminoarylthiazole derivative as described herein, in particular masitinib, or a pharmaceutically acceptable salt or solvate thereof, for use in the treatment of Alzheimer's disease in a patient having an ADAS-Cog score equal to or lower than 40 before treatment initiation, having an ADCS-ADL score equal to or greater, preferably greater, than 32 before treatment initiation, having a MMSE score equal to or greater than 13 before treatment initiation, and/or having a time from diagnosis to treatment initiation with masitinib, or a pharmaceutically acceptable salt or solvate thereof, equal to or less than 5 years. In other words, according to one embodiment, the present invention relates to a 2-aminoarylthiazole derivative as described herein, in particular masitinib, or a pharmaceutically acceptable salt or solvate thereof, for use in the treatment of Alzheimer's disease in a patient having a baseline ADAS-Cog score equal to or lower than 40, having a baseline ADCS-ADL score equal to or greater, preferably greater, than 32, having a baseline MMSE score equal to or greater than 13, and/or having a time from diagnosis to treatment initiation with masitinib, or a pharmaceutically acceptable salt or solvate thereof, equal to or less than 5 years.

According to one embodiment, the present invention relates to a 2-aminoarylthiazole derivative as described herein, in particular masitinib, or a pharmaceutically acceptable salt or solvate thereof, for use in the treatment of less severe Alzheimer's disease as defined herein in a patient in need thereof.

In one embodiment, the severity of Alzheimer's disease is determined using the Alzheimer's Disease Assessment Scale—Cognitive Subscale (ADAS-Cog), the Alzheimer's Disease Cooperative Study—Activities of Daily Living (ADCS-ADL) scale, and/or the Mini-Mental State Examination (MMSE).

In one embodiment, the severity of Alzheimer's disease is determined using the ADAS-Cog. The ADAS-Cog is widely used to determine the severity of cognitive symptoms of dementia. The ADAS-Cog examines 11 aspects of cognitive performance: spoken language, comprehension, word finding, word recall, naming, orientation, commands, ideational praxis, constructional praxis, word recognition, and recall instructions (Rosen et al., Am J Psychiatry. 1984 November; 141(11):1356-64). The ADAS-Cog score is based on the number of errors made in each assessed item, with a total score ranging from 0 to 70. A score of 70 represents the most severe cognitive impairment and a score of 0 represents the least cognitive impairment. Accordingly, an increase of the ADAS-Cog score over time, for example as compared to the ADAS-Cog score at baseline, corresponds to a worsening of cognitive functions and therefore to a progression of Alzheimer's disease. Conversely, a lack of increase (i.e., a stable ADAS-Cog score) or a decrease of the ADAS-Cog score over time, for example as compared to the ADAS-Cog score at baseline, corresponds to a stabilization of Alzheimer's disease. In one embodiment, an ADAS-Cog score equal to or lower than 40 corresponds to less severe Alzheimer's disease. In one embodiment, an ADAS-Cog score equal to or lower than 35 corresponds to less severe Alzheimer's disease.

In one embodiment, the severity of Alzheimer's disease is determined using the ADCS-ADL. The ADCS-ADL is widely used to assess the competence of patients with dementia, such as Alzheimer's disease, in basic and instrumental activities of daily living. The ADCS-ADL comprises 23 questions, with a total score ranging from 0 to 78 (Galasko et al., Alzheimer Dis Assoc Disord. 1997; 11 Suppl 2:S33-9). A score of 78 represents the least functional impairment and a score of 0 represents the most severe functional impairment. Accordingly, a decrease of the ADCS-ADL score over time, for example as compared to the ADCS-ADL score at baseline, corresponds to a worsening of the functional impairment and therefore to a progression of Alzheimer's disease. Conversely, a lack of decrease (i.e., a stable ADCS-ADL score) or an increase of the ADCS-ADL score over time, for example as compared to the ADCS-ADL score at baseline, corresponds to a stabilization of Alzheimer's disease. In one embodiment, an ADCS-ADL score equal to or greater, preferably greater, than 32 corresponds to less severe Alzheimer's disease. In one embodiment, an ADCS-ADL score equal to or greater, preferably greater, than 38 corresponds to less severe Alzheimer's disease.

In one embodiment, the severity of Alzheimer's disease is determined using the MMSE. The Mini-Mental State Examination (MMSE) is widely used to screen the cognitive impairments seen in dementia, with a focus on dementia associated with Alzheimer's disease. The MMSE comprises 11 items assessing orientation, registration, attention and calculation, recall, and language, with a total score ranging from 0 to 30 (Folstein et al., J Psychiatr Res. 1975 November; 12(3):189-98). A score of 30 represents the least cognitive impairment and a score of 0 represents the most severe cognitive impairment. Accordingly, a decrease of the MMSE score over time, for example as compared to the MMSE score at baseline, corresponds to a worsening of cognitive functions and therefore to a progression of Alzheimer's disease. Conversely, a lack of decrease (i.e., a stable MMSE score) or an increase of the MMSE score over time, for example as compared to the MMSE score at baseline, corresponds to a stabilization of Alzheimer's disease. In one embodiment, a MMSE score of 26 or more corresponds to normal cognitive functions, a MMSE score ranging from 21 to 25 corresponds to mild cognitive impairment, a MMSE score ranging from 12 to 20 corresponds to moderate cognitive impairment, and a MMSE score lower than 12 corresponds to severe cognitive impairment.

In one embodiment, a MMSE score equal to or greater than 13, preferably equal to or greater than 14 corresponds to less severe Alzheimer's disease. Thus, in one embodiment, a MMSE score equal to or greater than 14 excludes moderately severe and severe Alzheimer's disease. In one embodiment, a MMSE score ranging from 12 to 25, preferably from 13 to 25 corresponds to less severe Alzheimer's disease. In one embodiment, a MMSE score ranging from 21 to 25 corresponds to mildly severe Alzheimer's disease or mild Alzheimer's disease. In one embodiment, a MMSE score ranging from 12 to 20, preferably from 13 to 20, corresponds to moderately severe Alzheimer's disease or moderate Alzheimer's disease. In one embodiment, a MMSE score lower than 13, preferably lower than 12 corresponds to severe Alzheimer's disease.

In one embodiment, less severe Alzheimer's disease is defined as a baseline ADAS-Cog score equal to or lower than 40, a baseline ADCS-ADL score equal to or greater, preferably greater, than 32, and/or a baseline MMSE score equal to or greater than 13. Thus, in one embodiment, the present invention relates to a 2-aminoarylthiazole derivative as described herein, in particular masitinib, or a pharmaceutically acceptable salt or solvate thereof, for use in the treatment of less severe Alzheimer's disease in a patient in need thereof, said patient having a baseline ADAS-Cog score equal to or lower than 40, having a baseline ADCS-ADL score equal to or greater, preferably greater, than 32, and/or having a baseline MMSE score equal to or greater than 13.

In one embodiment, less severe Alzheimer's disease is defined as a baseline ADAS-Cog score equal to or lower than 40, and/or a baseline ADCS-ADL score equal to or greater, preferably greater, than 32. Thus, in one embodiment, the present invention relates to a 2-aminoarylthiazole derivative as described herein, in particular masitinib, or a pharmaceutically acceptable salt or solvate thereof, for use in the treatment of less severe Alzheimer's disease in a patient in need thereof, said patient having a baseline ADAS-Cog score equal to or lower than 40, and/or having a baseline ADCS-ADL score equal to or greater, preferably greater, than 32.

In one embodiment, less severe Alzheimer's disease is defined as a baseline ADAS-Cog score equal to or lower than 35, and/or a baseline ADCS-ADL score equal to or greater, preferably greater, than 38. Thus, in one embodiment, the present invention relates to a 2-aminoarylthiazole derivative as described herein, in particular masitinib, or a pharmaceutically acceptable salt or solvate thereof, for use in the treatment of less severe Alzheimer's disease in a patient in need thereof, said patient having a baseline ADAS-Cog score equal to or lower than 35, and/or having a baseline ADCS-ADL score equal to or greater, preferably greater, than 38.

According to one embodiment, the present invention relates to a 2-aminoarylthiazole derivative as described herein, in particular masitinib, or a pharmaceutically acceptable salt or solvate thereof, for use in the treatment of early-stage Alzheimer's disease as defined herein in a patient in need thereof.

In one embodiment, the stage of Alzheimer's disease is determined using the MMSE described above.

In one embodiment, a MMSE score equal to or greater than 12, 13, 14, 15, 16, 17, 18, 19, 20, or 21, preferably equal to or greater than 13, more preferably equal to or greater than 14, corresponds to early-stage Alzheimer's disease. In one embodiment, the present invention relates to a 2-aminoarylthiazole derivative as described herein, in particular masitinib, or a pharmaceutically acceptable salt or solvate thereof, for use in the treatment of early-stage Alzheimer's disease in a patient in need thereof, said patient having a baseline MMSE equal to or greater than 13, preferably equal to or greater than 14, more preferably equal to or greater than 21.

According to one embodiment, the present invention relates to a 2-aminoarylthiazole derivative as described herein, in particular masitinib, or a pharmaceutically acceptable salt or solvate thereof, for use in the treatment of early-stage Alzheimer's dementia as defined herein in a patient in need thereof.

In one embodiment, the stage of Alzheimer's dementia is determined using the ADCS-ADL scale described above, the MMSE described above, and/or the time from diagnosis to treatment initiation with the 2-aminoarylthiazole derivative as described herein, in particular masitinib, or a pharmaceutically acceptable salt or solvate thereof.

According to one embodiment, the stage of Alzheimer's dementia is determined using the ADCS-ADL scale described above. In one embodiment, an ADCS-ADL score equal to or greater, preferably greater, than 50 corresponds to early-stage Alzheimer's dementia. In one embodiment, an ADCS-ADL score equal to or greater, preferably greater, than 55 corresponds to early-stage Alzheimer's dementia.

According to one embodiment, the stage of Alzheimer's dementia is determined using the MMSE described above. In one embodiment, a MMSE score equal to or greater than 14 corresponds to early-stage Alzheimer's dementia. In one embodiment, a MMSE score ranging from 21 to 25 corresponds to early-stage Alzheimer's dementia.

According to one embodiment, the stage of Alzheimer's dementia is determined based on the time from diagnosis to treatment initiation with the 2-aminoarylthiazole derivative as described herein, in particular masitinib, or a pharmaceutically acceptable salt or solvate thereof. In one embodiment, a time from diagnosis to treatment initiation with the 2-aminoarylthiazole derivative as described herein, in particular masitinib, or a pharmaceutically acceptable salt or solvate thereof, equal to or less than 3 years corresponds to early-stage Alzheimer's dementia. In one embodiment, a time from diagnosis to treatment initiation with the 2-aminoarylthiazole derivative as described herein, in particular masitinib, or a pharmaceutically acceptable salt or solvate thereof, equal to or less than 2 years corresponds to early-stage Alzheimer's dementia.

According to one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score as defined above, and/or having a baseline MMSE score as defined above.

In one embodiment, early-stage Alzheimer's dementia is defined as a baseline ADCS-ADL score equal to or greater, preferably greater, than 50, and/or a baseline MMSE score equal to or greater than 14, or ranging from 21 to 25. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 50, and/or having a baseline MMSE score equal to or greater than 14, or ranging from 21 to 25.

In one embodiment, early-stage Alzheimer's dementia is defined as a baseline ADCS-ADL score equal to or greater, preferably greater, than 55, and/or a baseline MMSE score equal to or greater than 14, or ranging from 21 to 25. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 55, and/or having a baseline MMSE score equal to or greater than 14, or ranging from 21 to 25.

According to one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score as defined above, and/or having a time from diagnosis to treatment initiation with the 2-aminoarylthiazole derivative as described herein, in particular masitinib, or a pharmaceutically acceptable salt or solvate thereof, as defined above.

In one embodiment, early-stage Alzheimer's dementia is defined as a baseline ADCS-ADL score equal to or greater, preferably greater, than 50, and/or a time from diagnosis to treatment initiation with the 2-aminoarylthiazole derivative as described herein, in particular masitinib, or a pharmaceutically acceptable salt or solvate thereof, equal to or less than 3 years, or equal to or less than 2 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 50, and/or having a time from diagnosis to treatment initiation with the 2-aminoarylthiazole derivative as described herein, in particular masitinib, or a pharmaceutically acceptable salt or solvate thereof, equal to or less than 3 years, or equal to or less than 2 years.

In one embodiment, early-stage Alzheimer's dementia is defined as a baseline ADCS-ADL score equal to or greater, preferably greater, than 55, and/or a time from diagnosis to treatment initiation with the 2-aminoarylthiazole derivative as described herein, in particular masitinib, or a pharmaceutically acceptable salt or solvate thereof, equal to or less than 3 years, or equal to or less than 2 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 55, and/or having a time from diagnosis to treatment initiation with the 2-aminoarylthiazole derivative as described herein, in particular masitinib, or a pharmaceutically acceptable salt or solvate thereof, equal to or less than 3 years, or equal to or less than 2 years.

According to one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline MMSE score as defined above, and/or having a time from diagnosis to treatment initiation with the 2-aminoarylthiazole derivative as described herein, in particular masitinib, or a pharmaceutically acceptable salt or solvate thereof, as defined above.

In one embodiment, early-stage Alzheimer's dementia is defined as a baseline MMSE score equal to or greater than 14, and/or a time from diagnosis to treatment initiation with the 2-aminoarylthiazole derivative as described herein, in particular masitinib, or a pharmaceutically acceptable salt or solvate thereof, equal to or less than 3 years, or equal to or less than 2 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline MMSE score equal to or greater than 14, and/or having a time from diagnosis to treatment initiation with the 2-aminoarylthiazole derivative as described herein, in particular masitinib, or a pharmaceutically acceptable salt or solvate thereof, equal to or less than 3 years, or equal to or less than 2 years.

In one embodiment, early-stage Alzheimer's dementia is defined as a baseline MMSE score ranging from 21 to 25, and/or having a time from diagnosis to treatment initiation with the 2-aminoarylthiazole derivative as described herein, in particular masitinib, or a pharmaceutically acceptable salt or solvate thereof, equal to or less than 3 years, or equal to or less than 2 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline MMSE score ranging from 21 to 25, and/or having a time from diagnosis to treatment initiation with the 2-aminoarylthiazole derivative as described herein, in particular masitinib, or a pharmaceutically acceptable salt or solvate thereof, equal to or less than 3 years, or equal to or less than 2 years.

According to one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score as defined above, having a baseline MMSE score as defined above, and/or having a time from diagnosis to treatment initiation with the 2-aminoarylthiazole derivative as described herein, in particular masitinib, or a pharmaceutically acceptable salt or solvate thereof, as defined above.

In one embodiment, early-stage Alzheimer's dementia is defined as a baseline ADCS-ADL score equal to or greater, preferably greater, than 50, a baseline MMSE score equal to or greater than 14, and/or a time from diagnosis to treatment initiation with the 2-aminoarylthiazole derivative as described herein, in particular masitinib, or a pharmaceutically acceptable salt or solvate thereof, equal to or less than 3 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 50, having a baseline MMSE score equal to or greater than 14, and/or having a time from diagnosis to treatment initiation with the 2-aminoarylthiazole derivative as described herein, in particular masitinib, or a pharmaceutically acceptable salt or solvate thereof, equal to or less than 3 years.

In one embodiment, early-stage Alzheimer's dementia is defined as a baseline ADCS-ADL score equal to or greater, preferably greater, than 50, a baseline MMSE score equal to or greater than 14, and/or a time from diagnosis to treatment initiation with the 2-aminoarylthiazole derivative as described herein, in particular masitinib, or a pharmaceutically acceptable salt or solvate thereof, equal to or less than 2 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 50, having a baseline MMSE score equal to or greater than 14, and/or having a time from diagnosis to treatment initiation with the 2-aminoarylthiazole derivative as described herein, in particular masitinib, or a pharmaceutically acceptable salt or solvate thereof, equal to or less than 2 years.

In one embodiment, early-stage Alzheimer's dementia is defined as a baseline ADCS-ADL score equal to or greater, preferably greater, than 50, a baseline MMSE score ranging from 21 to 25, and/or a time from diagnosis to treatment initiation with the 2-aminoarylthiazole derivative as described herein, in particular masitinib, or a pharmaceutically acceptable salt or solvate thereof, equal to or less than 3 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 50, having a baseline MMSE score ranging from 21 to 25, and/or having a time from diagnosis to treatment initiation with the 2-aminoarylthiazole derivative as described herein, in particular masitinib, or a pharmaceutically acceptable salt or solvate thereof, equal to or less than 3 years.

In one embodiment, early-stage Alzheimer's dementia is defined as a baseline ADCS-ADL score equal to or greater, preferably greater, than 50, a baseline MMSE score ranging from 21 to 25, and/or a time from diagnosis to treatment initiation with the 2-aminoarylthiazole derivative as described herein, in particular masitinib, or a pharmaceutically acceptable salt or solvate thereof, equal to or less than 2 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 50, having a baseline MMSE score ranging from 21 to 25, and/or having a time from diagnosis to treatment initiation with the 2-aminoarylthiazole derivative as described herein, in particular masitinib, or a pharmaceutically acceptable salt or solvate thereof, equal to or less than 2 years.

In one embodiment, early-stage Alzheimer's dementia is defined as a baseline ADCS-ADL score equal to or greater, preferably greater, than 55, a baseline MMSE score equal to or greater than 14, and/or a time from diagnosis to treatment initiation with the 2-aminoarylthiazole derivative as described herein, in particular masitinib, or a pharmaceutically acceptable salt or solvate thereof, equal to or less than 3 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 55, having a baseline MMSE score equal to or greater than 14, and/or having a time from diagnosis to treatment initiation with the 2-aminoarylthiazole derivative as described herein, in particular masitinib, or a pharmaceutically acceptable salt or solvate thereof, equal to or less than 3 years.

In one embodiment, early-stage Alzheimer's dementia is defined as a baseline ADCS-ADL score equal to or greater, preferably greater, than 55, a baseline MMSE score equal to or greater than 14, and/or a time from diagnosis to treatment initiation with the 2-aminoarylthiazole derivative as described herein, in particular masitinib, or a pharmaceutically acceptable salt or solvate thereof, equal to or less than 2 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 55, having a baseline MMSE score equal to or greater than 14, and/or having a time from diagnosis to treatment initiation with the 2-aminoarylthiazole derivative as described herein, in particular masitinib, or a pharmaceutically acceptable salt or solvate thereof, equal to or less than 2 years.

In one embodiment, early-stage Alzheimer's dementia is defined as a baseline ADCS-ADL score equal to or greater, preferably greater, than 55, a baseline MMSE score ranging from 21 to 25, and/or a time from diagnosis to treatment initiation with the 2-aminoarylthiazole derivative as described herein, in particular masitinib, or a pharmaceutically acceptable salt or solvate thereof, equal to or less than 3 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 55, having a baseline MMSE score ranging from 21 to 25, and/or having a time from diagnosis to treatment initiation with the 2-aminoarylthiazole derivative as described herein, in particular masitinib, or a pharmaceutically acceptable salt or solvate thereof, equal to or less than 3 years.

In one embodiment, early-stage Alzheimer's dementia is defined as a baseline ADCS-ADL score equal to or greater, preferably greater, than 55, a baseline MMSE score ranging from 21 to 25, and/or a time from diagnosis to treatment initiation with the 2-aminoarylthiazole derivative as described herein, in particular masitinib, or a pharmaceutically acceptable salt or solvate thereof, equal to or less than 2 years. Thus, in one embodiment, the present invention relates to a 2-aminoarylthiazole derivative as described herein, in particular masitinib, or a pharmaceutically acceptable salt or solvate thereof, for use in the treatment of early-stage Alzheimer's dementia in a patient in need thereof, said patient having a baseline ADCS-ADL score equal to or greater, preferably greater, than 55, having a baseline MMSE score ranging from 21 to 25, and/or having a time from diagnosis to treatment initiation with the 2-aminoarylthiazole derivative as described herein, in particular masitinib, or a pharmaceutically acceptable salt or solvate thereof, equal to or less than 2 years.

In one embodiment, the AD patient to be treated according to the present invention has a baseline ADAS-Cog score equal to or lower than 60, 55, 50, 45, 40, 35, 30, 25, 20, 15, or 10. In one embodiment, the AD patient to be treated according to the present invention has a baseline ADAS-Cog score equal to or lower than 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10. In one embodiment, the AD patient to be treated according to the present invention has a baseline ADAS-Cog score equal to or lower than 40, 35, 32, or 25.

In one embodiment, the AD patient to be treated according to the present invention has a baseline ADCS-ADL score equal to or greater, preferably greater, than 20, 25, 30, 35, 39, 45, 50, 55, 60, 65, or 70. In one embodiment, the AD patient to be treated according to the present invention has a baseline ADCS-ADL score equal to or greater, preferably greater, than 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70. In one embodiment, the AD patient to be treated according to the present invention has a baseline ADCS-ADL score equal to or greater, preferably greater than 32, 35, 38, 39, 41, 47, or 55. In one embodiment, the AD patient to be treated according to the present invention has a baseline ADCS-ADL score equal to or greater, preferably greater, than 38, 39, 50, or 55. In one embodiment, the AD patient to be treated according to the present invention has a baseline ADCS-ADL score equal to or greater than 38 or 50, or greater than 39 or 55. In one embodiment, the AD patient to be treated according to the present invention has a baseline ADCS-ADL score equal to or greater, preferably greater, than 50. In one embodiment, the AD patient to be treated according to the present invention has a baseline ADCS-ADL score equal to or greater, preferably greater, than 55.

In one embodiment, the AD patient to be treated according to the present invention has a baseline MMSE score equal to or greater than 12, 13, 14, 15, 16, 17, 18, 19, 20, or 21. In one embodiment, the AD patient to be treated according to the present invention has a baseline MMSE score equal to or greater than 13, preferably equal to or greater than 14. In one embodiment, the AD patient to be treated according to the present invention has a baseline MMSE score ranging from 12 to 20. In one embodiment, the AD patient to be treated according to the present invention has a baseline MMSE score ranging from 21 to 25.

In one embodiment, the AD patient to be treated according to the present invention has a time from diagnosis to treatment initiation with the 2-aminoarylthiazole derivative as described herein, in particular masitinib, or a pharmaceutically acceptable salt or solvate thereof, equal to or less than 10, 9.5, 9, 8.5, 8, 7.5, 7, 6.5, 6, 5.5, 5, 4.5, 4, 3.5, 3, 2.5, 2 years, 1.5, or 1 year(s). In one embodiment, the AD patient to be treated according to the present invention has a time from diagnosis to treatment initiation with the 2-aminoarylthiazole derivative as described herein, in particular masitinib, or a pharmaceutically acceptable salt or solvate thereof, equal to or less than 5, 4, 3, or 2 years. In one embodiment, the AD patient to be treated according to the present invention has a time from diagnosis to treatment initiation with the 2-aminoarylthiazole derivative as described herein, in particular masitinib, or a pharmaceutically acceptable salt or solvate thereof, equal to or less than 3 years, preferably equal to or less than 2 years.

In one embodiment, the AD patient to be treated according to the present invention has a baseline ADAS-Cog score equal to or lower than 60, 55, 50, 45, 40, 35, 30, 25, 20, 15, or 10, and/or has a baseline ADCS-ADL score greater than 20, 25, 30, 35, 39, 45, 50, 55, 60, 65, or 70. In one embodiment, the AD patient to be treated according to the present invention has a baseline ADAS-Cog score equal to or lower than 45, 44, 43, 42, 41, 40, 39, 38, 37, 36, 35, 34, 33, 32, 31, 30, 29, 28, 27, 26, or 25, and/or has a baseline ADCS-ADL score equal to or greater, preferably greater, than 30, 31, 32, 33, 34, 35, 36, 37, 38, 39, 40, 41, 42, 43, 44, 45, 46, 47, 48, 49, 50, 51, 52, 53, 54, or 55. In one embodiment, the AD patient to be treated according to the present invention has a baseline ADAS-Cog score equal to or lower than 40, 35, 32, or 25, and/or has a baseline ADCS-ADL score equal to or greater, preferably greater, than 38, 39, 50 or 55. In one embodiment, the AD patient to be treated according to the present invention has a baseline ADAS-Cog score equal to or lower than 40, 35, 32, or 25, and/or has a baseline ADCS-ADL score equal to or greater, preferably greater, than 50. In one embodiment, the AD patient to be treated according to the present invention has a baseline ADAS-Cog score equal to or lower than 40, 35, 32, or 25, and/or has a baseline ADCS-ADL score equal to or greater, preferably greater, than 55.

In one embodiment, the present invention relates to a 2-aminoarylthiazole derivative as described herein, in particular masitinib, or a pharmaceutically acceptable salt or solvate thereof, for use in the treatment of AD in a patient having a baseline ADAS-Cog score equal to or lower than 40, 35, 32, or 25, and/or having a baseline ADCS-ADL score equal to or greater than 38 or 50, or greater than 39 or 55.

In one embodiment, the AD patient to be treated according to the present invention has a baseline ADAS-Cog score equal to or lower than 60, 55, 50, 45, 40, 35, 30, 25, 20, 15, or 10, and/or has a baseline MMSE score equal to or greater than 12, 13, 14, 15, 16, 17, 18, 19, 20, or 21. In one embodiment, the AD patient to be treated according to the present invention has a baseline ADAS-Cog score equal to or lower than 45, 44, 43, 42, 41, 40, 39, 38, 37, 36, 35, 34, 33, 32, 31, 30, 29, 28, 27, 26, or 25, and/or has a baseline MMSE score equal to or greater than 13, 14, 15, 16, 17, 18, 19, or 20. In one embodiment, the AD patient to be treated according to the present invention has a baseline ADAS-Cog score equal to or lower than 40, 35, 32, or 25, and/or has a baseline MMSE score equal to or greater than 13, preferably equal to or greater than 14, or ranging from 21 to 25. In one embodiment, the AD patient to be treated according to the present invention has a baseline ADAS-Cog score equal to or lower than 40, 35, 32, or 25, and/or has a baseline MMSE score equal to or greater than 14. In one embodiment, the AD patient to be treated according to the present invention has a baseline ADAS-Cog score equal to or lower than 40, 35, 32, or 25, and/or has a baseline MMSE score ranging from 21 to 25.

In one embodiment, the AD patient to be treated according to the present invention has a baseline ADAS-Cog score equal to or lower than 60, 55, 50, 45, 40, 35, 30, 25, 20, 15, or 10, and/or has a time from diagnosis to treatment initiation with the 2-aminoarylthiazole derivative as described herein, in particular masitinib, or a pharmaceutically acceptable salt or solvate thereof, equal to or less than 10, 9.5, 9, 8.5, 8, 7.5, 7, 6.5, 6, 5.5, 5, 4.5, 4, 3.5, 3, 2.5, 2, 1.5, or 1 year(s). In one embodiment, the AD patient to be treated according to the present invention has a baseline ADAS-Cog score equal to or lower than 45, 44, 43, 42, 41, 40, 39, 38, 37, 36, 35, 34, 33, 32, 31, 30, 29, 28, 27, 26, or 25, and/or has a time from diagnosis to treatment initiation with the 2-aminoarylthiazole derivative as described herein, in particular masitinib, or a pharmaceutically acceptable salt or solvate thereof, equal to or less than 5, 4.5, 4, 3.5, 3, 2.5, or 2 years. In one embodiment, the AD patient to be treated according to the present invention has a baseline ADAS-Cog score equal to or lower than 40, 35, 32, or 25, and/or has a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years. In one embodiment, the AD patient to be treated according to the present invention has a baseline ADAS-Cog score equal to or lower than 40, 35, 32, or 25, and/or has a time from diagnosis to treatment initiation with the 2-aminoarylthiazole derivative as described herein, in particular masitinib, or a pharmaceutically acceptable salt or solvate thereof, equal to or less than 3 years. In one embodiment, the AD patient to be treated according to the present invention has a baseline ADAS-Cog score equal to or lower than 40, 35, 32, or 25, and/or has a time from diagnosis to treatment initiation with the 2-aminoarylthiazole derivative as described herein, in particular masitinib, or a pharmaceutically acceptable salt or solvate thereof, equal to or less than 2 years.

In one embodiment, the AD patient to be treated according to the present invention has a baseline ADCS-ADL score equal to or greater, preferably greater, than 20, 25, 30, 35, 39, 45, 50, 55, 60, 65, or 70, and/or has a baseline MMSE score equal to or greater than 12, 13, 14, 15, 16, 17, 18, 19, 20, or 21. In one embodiment, the AD patient to be treated according to the present invention has a baseline ADCS-ADL score equal to or greater, preferably greater, than 30, 31, 32, 33, 34, 35, 36, 37, 38, 39, 40, 41, 42, 43, 44, 45, 46, 47, 48, 49, 50, 51, 52, 53, 54, or 55, and/or has a baseline MMSE score equal to or greater than 13, 14, 15, 16, or 17. In one embodiment, the AD patient to be treated according to the present invention has a baseline ADCS-ADL score equal to or greater, preferably greater, than 38, 39, 50 or 55, and/or has a baseline MMSE score equal to or greater than 13, preferably equal to or greater than 14, or ranging from 21 to 25.

In one embodiment, the present invention relates to a 2-aminoarylthiazole derivative as described herein, in particular masitinib, or a pharmaceutically acceptable salt or solvate thereof, for use in the treatment of AD in a patient having a baseline ADCS-ADL score equal to or greater than 38 or 50, or greater than 39 or 55, and/or having a baseline MMSE score equal to or greater than 13, preferably equal to or greater than 14, or ranging from 21 to 25.

In one embodiment, the AD patient to be treated according to the present invention has a baseline ADCS-ADL score greater than 20, 25, 30, 35, 39, 45, 50, 55, 60, 65, or 70, and/or has a time from diagnosis to treatment initiation with the 2-aminoarylthiazole derivative as described herein, in particular masitinib, or a pharmaceutically acceptable salt or solvate thereof, equal to or less than 10, 9.5, 9, 8.5, 8, 7.5, 7, 6.5, 6, 5.5, 5, 4.5, 4, 3.5, 3, 2.5, 2, 1.5, or 1 year(s). In one embodiment, the AD patient to be treated according to the present invention has a baseline ADCS-ADL score equal to or greater, preferably greater, than 30, 31, 32, 33, 34, 35, 36, 37, 38, 39, 40, 41, 42, 43, 44, 45, 46, 47, 48, 49, 50, 51, 52, 53, 54, or 55, and/or has a time from diagnosis to treatment initiation with the 2-aminoarylthiazole derivative as described herein, in particular masitinib, or a pharmaceutically acceptable salt or solvate thereof, equal to or less than 5, 4.5, 4, 3.5, 3, 2.5, or 2 years. In one embodiment, the AD patient to be treated according to the present invention has a baseline ADCS-ADL score equal to or greater, preferably greater, than 38, 39, 50, or 55, and/or has a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years.

In one embodiment, the present invention relates to a 2-aminoarylthiazole derivative as described herein, in particular masitinib, or a pharmaceutically acceptable salt or solvate thereof, for use in the treatment of AD in a patient having a baseline ADCS-ADL score equal to or greater than 38 or 50, or greater than 39 or 55, and having a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years.

In one embodiment, the AD patient to be treated according to the present invention has a baseline MMSE score equal to or greater than 12, 13, 14, 15, 16, 17, 18, 19, 20, or 21, and/or has a time from diagnosis to treatment initiation with the 2-aminoarylthiazole derivative as described herein, in particular masitinib, or a pharmaceutically acceptable salt or solvate thereof, equal to or less than 10, 9.5, 9, 8.5, 8, 7.5, 7, 6.5, 6, 5.5, 5, 4.5, 4, 3.5, 3, 2.5, 2, 1.5, or 1 year(s). In one embodiment, the AD patient to be treated according to the present invention has a baseline MMSE score equal to or greater than equal to or greater than 13, 14, 15, 16, or 17, and/or has a time from diagnosis to treatment initiation with the 2-aminoarylthiazole derivative as described herein, in particular masitinib, or a pharmaceutically acceptable salt or solvate thereof, equal to or less than 5, 4.5, 4, 3.5, 3, 2.5, or 2 years. In one embodiment, the AD patient to be treated according to the present invention has a baseline MMSE score equal to or greater than 13, preferably equal to or greater than 14, or ranging from 21 to 25, and/or has a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years, more preferably equal to or less than 2 years.

In one embodiment, the AD patient to be treated according to the present invention has a baseline ADAS-Cog score equal to or lower than 60, 55, 50, 45, 40, 35, 30, 25, 20, 15, or 10, has a baseline ADCS-ADL score equal to or greater, preferably greater, than 20, 25, 30, 35, 39, 45, 50, 55, 60, 65, or 70, and/or has a baseline MMSE score equal to or greater than 12, 13, 14, 15, 16, 17, 18, 19, 20, or 21. In one embodiment, the AD patient to be treated according to the present invention has a baseline ADAS-Cog score equal to or lower than 45, 44, 43, 42, 41, 40, 39, 38, 37, 36, 35, 34, 33, 32, 31, 30, 29, 28, 27, 26, or 25, has a baseline ADCS-ADL score equal to or greater, preferably greater, than 30, 31, 32, 33, 34, 35, 36, 37, 38, 39, 40, 41, 42, 43, 44, 45, 46, 47, 48, 49, 50, 51, 52, 53, 54, or 55, and/or has a baseline MMSE score equal to or greater than 13, 14, 15, 16, or 17. In one embodiment, the AD patient to be treated according to the present invention has a baseline ADAS-Cog score equal to or lower than 40, 35, 32, or 25, has a baseline ADCS-ADL score equal to or greater, preferably greater, than 38, 39, 50, or 55, and/or has a baseline MMSE score equal to or greater than 13, preferably equal to or greater than 14, or ranging from 21 to 25. In one embodiment, the AD patient to be treated according to the present invention has a baseline ADAS-Cog score equal to or lower than 25, has a baseline ADCS-ADL score equal to or greater, preferably greater, than 50, and/or has a baseline MMSE score equal to or greater than 14, preferably ranging from 21 to 25. In one embodiment, the AD patient to be treated according to the present invention has a baseline ADAS-Cog score equal to or lower than 25, has a baseline ADCS-ADL score equal to or greater, preferably greater, than 55, and/or has a baseline MMSE score equal to or greater than 14, preferably ranging from 21 to 25

In one embodiment, the present invention relates to a 2-aminoarylthiazole derivative as described herein, in particular masitinib, or a pharmaceutically acceptable salt or solvate thereof, for use in the treatment of AD in a patient having a baseline ADAS-Cog score equal to or lower than 40, 35, 32, or 25, having a baseline ADCS-ADL score equal to or greater than 38 or 50, or greater than 39 or 55, and/or having a baseline MMSE score equal to or greater than 13, preferably equal to or greater than 14, or ranging from 21 to 25.

In one embodiment, the AD patient to be treated according to the present invention has a baseline ADAS-Cog score equal to or lower than 60, 55, 50, 45, 40, 35, 30, 25, 20, 15, or 10, has a baseline ADCS-ADL score equal to or greater, preferably greater, than 20, 25, 30, 35, 39, 45, 50, 55, 60, 65, or 70, and/or has a time from diagnosis to treatment initiation with the 2-aminoarylthiazole derivative as described herein, in particular masitinib, or a pharmaceutically acceptable salt or solvate thereof, equal to or less than 10, 9.5, 9, 8.5, 8, 7.5, 7, 6.5, 6, 5.5, 5, 4.5, 4, 3.5, 3, 2.5, 2, 1.5, or 1 year(s). In one embodiment, the AD patient to be treated according to the present invention has a baseline ADAS-Cog score equal to or lower than 45, 44, 43, 42, 41, 40, 39, 38, 37, 36, 35, 34, 33, 32, 31, 30, 29, 28, 27, 26, or 25, has a baseline ADCS-ADL score equal to or greater, preferably greater, than 30, 31, 32, 33, 34, 35, 36, 37, 38, 39, 40, 41, 42, 43, 44, 45, 46, 47, 48, 49, 50, 51, 52, 53, 54, or 55, and/or has a time from diagnosis to treatment initiation with the 2-aminoarylthiazole derivative as described herein, in particular masitinib, or a pharmaceutically acceptable salt or solvate thereof, equal to or less than 5, 4.5, 4, 3.5, 3, 2.5, or 2 years. In one embodiment, the AD patient to be treated according to the present invention has a baseline ADAS-Cog score equal to or lower than 40, 35, 32, or 25, has a baseline ADCS-ADL score equal to or greater, preferably greater, than 38, 39, 50, or 55, and/or has a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years. In one embodiment, the AD patient to be treated according to the present invention has a baseline ADAS-Cog score equal to or lower than 25, has a baseline ADCS-ADL score equal to or greater, preferably greater, than 50, and/or has a time from diagnosis to treatment initiation with the 2-aminoarylthiazole derivative as described herein, in particular masitinib, or a pharmaceutically acceptable salt or solvate thereof, equal to or less than 3 years, preferably equal to or less than 2 years. In one embodiment, the AD patient to be treated according to the present invention has a baseline ADAS-Cog score equal to or lower than 25, has a baseline ADCS-ADL score equal to or greater, preferably greater, than 55, and/or has a time from diagnosis to treatment initiation with the 2-aminoarylthiazole derivative as described herein, in particular masitinib, or a pharmaceutically acceptable salt or solvate thereof, equal to or less than 3 years, preferably equal to or less than 2 years.

In one embodiment, the present invention relates to a 2-aminoarylthiazole derivative as described herein, in particular masitinib, or a pharmaceutically acceptable salt or solvate thereof, for use in the treatment of AD in a patient having a baseline ADAS-Cog score equal to or lower than 40, 35, 32, or 25, having a baseline ADCS-ADL score equal to or greater than 38 or 50, or greater than 39 or 55, and/or having a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years.

In one embodiment, the AD patient to be treated according to the present invention has a baseline ADAS-Cog score equal to or lower than 60, 55, 50, 45, 40, 35, 30, 25, 20, 15, or 10, has a baseline MMSE score equal to or greater than 12, 13, 14, 15, 16, 17, 18, 19, 20, or 21, and/or has a time from diagnosis to treatment initiation with the 2-aminoarylthiazole derivative as described herein, in particular masitinib, or a pharmaceutically acceptable salt or solvate thereof, equal to or less than 10, 9.5, 9, 8.5, 8, 7.5, 7, 6.5, 6, 5.5, 5, 4.5, 4, 3.5, 3, 2.5, 2, 1.5, or 1 year(s). In one embodiment, the AD patient to be treated according to the present invention has a baseline ADAS-Cog score equal to or lower than 45, 44, 43, 42, 41, 40, 39, 38, 37, 36, 35, 34, 33, 32, 31, 30, 29, 28, 27, 26, or 25, has a baseline MMSE score equal to or greater than 13, 14, 15, 16, or 17, and/or has a time from diagnosis to treatment initiation with the 2-aminoarylthiazole derivative as described herein, in particular masitinib, or a pharmaceutically acceptable salt or solvate thereof, equal to or less than 5, 4.5, 4, 3.5, 3, 2.5, or 2 years. In one embodiment, the AD patient to be treated according to the present invention has a baseline ADAS-Cog score equal to or lower than 40, 35, 32 or 25, has a baseline MMSE score equal to or greater than 13, preferably equal to or greater than 14, or ranging from 21 to 25, and/or has a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years. In one embodiment, the AD patient to be treated according to the present invention has a baseline ADAS-Cog score equal to or lower than 25, has a baseline MMSE score equal to or greater than 14, and/or has a time from diagnosis to treatment initiation with the 2-aminoarylthiazole derivative as described herein, in particular masitinib, or a pharmaceutically acceptable salt or solvate thereof, equal to or less than 3 years, preferably equal to or less than 2 years. In one embodiment, the AD patient to be treated according to the present invention has a baseline ADAS-Cog score equal to or lower than 25, has a baseline MMSE score ranging from 21 to 25, and/or has a time from diagnosis to treatment initiation with the 2-aminoarylthiazole derivative as described herein, in particular masitinib, or a pharmaceutically acceptable salt or solvate thereof, equal to or less than 3 years, preferably equal to or less than 2 years.

In one embodiment, the AD patient to be treated according to the present invention has a baseline ADCS-ADL score equal to or greater, preferably greater, than 20, 25, 30, 35, 39, 45, 50, 55, 60, 65, or 70, has a baseline MMSE score equal to or greater than 12, 13, 14, 15, 16, 17, 18, 19, 20, or 21, and/or has a time from diagnosis to treatment initiation with the 2-aminoarylthiazole derivative as described herein, in particular masitinib, or a pharmaceutically acceptable salt or solvate thereof, equal to or less than 10, 9.5, 9, 8.5, 8, 7.5, 7, 6.5, 6, 5.5, 5, 4.5, 4, 3.5, 3, 2.5, 2, 1.5, or 1 year(s). In one embodiment, the AD patient to be treated according to the present invention has a baseline ADCS-ADL score equal to or greater, preferably greater, than 45, 44, 43, 42, 41, 40, 39, 38, 37, 36, 35, 34, 33, 32, 31, 30, 29, 28, 27, 26, or 25, has a baseline MMSE score equal to or greater than 13, 14, 15, 16, or 17, and/or has a time from diagnosis to treatment initiation with the 2-aminoarylthiazole derivative as described herein, in particular masitinib, or a pharmaceutically acceptable salt or solvate thereof, equal to or less than 5, 4.5, 4, 3.5, 3, 2.5, or 2 years. In one embodiment, the AD patient to be treated according to the present invention has a baseline ADCS-ADL score equal to or greater, preferably greater, than 38, 39, 50, or 55, has a baseline MMSE score equal to or greater than 13, preferably equal to or greater than 14, or ranging from 21 to 25, and/or has a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years.

In one embodiment, the present invention relates to a 2-aminoarylthiazole derivative as described herein, in particular masitinib, or a pharmaceutically acceptable salt or solvate thereof, for use in the treatment of AD in a patient having a baseline ADCS-ADL score equal to or greater than 38 or 50, or greater than 39 or 55, having a baseline MMSE score equal to or greater than 13, preferably equal to or greater than 14, or ranging from 21 to 25, and/or having a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years.

In one embodiment, the AD patient to be treated according to the present invention has a baseline ADAS-Cog score equal to or lower than 60, 55, 50, 45, 40, 35, 30, 25, 20, 15, or 10, has a baseline ADCS-ADL score equal to or greater, preferably greater, than 20, 25, 30, 35, 39, 45, 50, 55, 60, 65, or 70, has a baseline MMSE score equal to or greater than 12, 13, 14, 15, 16, 17, 18, 19, 20, or 21, and/or has a time from diagnosis to treatment initiation with the 2-aminoarylthiazole derivative as described herein, in particular masitinib, or a pharmaceutically acceptable salt or solvate thereof, equal to or less than 10, 9.5, 9, 8.5, 8, 7.5, 7, 6.5, 6, 5.5, 5, 4.5, 4, 3.5, 3, 2.5, 2, 1.5, or 1 year(s). In one embodiment, the AD patient to be treated according to the present invention has a baseline ADAS-Cog score equal to or lower than 45, 44, 43, 42, 41, 40, 39, 38, 37, 36, 35, 34, 33, 32, 31, 30, 29, 28, 27, 26, or 25, has a baseline ADCS-ADL score equal to or greater, preferably greater, than 30, 31, 32, 33, 34, 35, 36, 37, 38, 39, 40, 41, 42, 43, 44, 45, 46, 47, 48, 49, 50, 51, 52, 53, 54, or 55, has a baseline MMSE score equal to or greater than 13, 14, 15, 16, or 17, and/or has a time from diagnosis to treatment initiation with the 2-aminoarylthiazole derivative as described herein, in particular masitinib, or a pharmaceutically acceptable salt or solvate thereof, equal to or less than 5, 4.5, 4, 3.5, 3, 2.5, or 2 years. In one embodiment, the AD patient to be treated according to the present invention has a baseline ADAS-Cog score equal to or lower than 40, 35, 32 or 25, has a baseline ADCS-ADL score equal to or greater, preferably greater, than 38, 39, 50, or 55, has a baseline MMSE score equal to or greater than 13, preferably equal to or greater than 14, or ranging from 21 to 25, and/or has a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years. In one embodiment, the AD patient to be treated according to the present invention has a baseline ADAS-Cog score equal to or lower than 25, has a baseline ADCS-ADL score equal to or greater, preferably greater, than 50, has a baseline MMSE score equal to or greater than 14, preferably ranging from 21 to 25, and/or has a time from diagnosis to treatment initiation with the 2-aminoarylthiazole derivative as described herein, in particular masitinib, or a pharmaceutically acceptable salt or solvate thereof, equal to or less than 3 years. In one embodiment, the AD patient to be treated according to the present invention has a baseline ADAS-Cog score equal to or lower than 25, has a baseline ADCS-ADL score equal to or greater, preferably greater, than 55, has a baseline MMSE score equal to or greater than 14, preferably ranging from 21 to 25, and/or has a time from diagnosis to treatment initiation with the 2-aminoarylthiazole derivative as described herein, in particular masitinib, or a pharmaceutically acceptable salt or solvate thereof, equal to or less than 3 years. In one embodiment, the AD patient to be treated according to the present invention has a baseline ADAS-Cog score equal to or lower than 25, has a baseline ADCS-ADL score equal to or greater, preferably greater, than 50, has a baseline MMSE score equal to or greater than 14, preferably ranging from 21 to 25, and/or has a time from diagnosis to treatment initiation with the 2-aminoarylthiazole derivative as described herein, in particular masitinib, or a pharmaceutically acceptable salt or solvate thereof, equal to or less than 2 years. In one embodiment, the AD patient to be treated according to the present invention has a baseline ADAS-Cog score equal to or lower than 25, has a baseline ADCS-ADL score equal to or greater, preferably greater, than 55, has a baseline MMSE score equal to or greater than 14, preferably ranging from 21 to 25, and/or has a time from diagnosis to treatment initiation with the 2-aminoarylthiazole derivative as described herein, in particular masitinib, or a pharmaceutically acceptable salt or solvate thereof, equal to or less than 2 years.

In one embodiment, the present invention relates to a 2-aminoarylthiazole derivative as described herein, in particular masitinib, or a pharmaceutically acceptable salt or solvate thereof, for use in the treatment of AD in a patient having a baseline ADAS-Cog score equal to or lower than 40, 35, 32, or 25, having a baseline ADCS-ADL score equal to or greater 38 or 50, or greater than 39 or 55, having a baseline MMSE score equal to or greater than 13, preferably equal to or greater than 14, or ranging from 21 to 25, and having a time from diagnosis to treatment initiation with the 2-aminoarylthiazole derivative as described herein, in particular masitinib, or a pharmaceutically acceptable salt or solvate thereof, equal to or less than 5, 4, 3, or 2 years, preferably equal to or less than 3 years.

In one embodiment, the patient is 50-year-old, 55-year-old, 60-year-old, 65-year-old, 70-year-old, 75-year-old, 80-year-old, or older. In one embodiment, the patient is 50-year-old, or older. In one embodiment, the patient is at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 years of age.

In one embodiment, the patient is younger than 95, 90, 85, 80, 75, or 70 years of age.

In one embodiment, the patient is male. In one embodiment, the patient is female.

In one embodiment, the patient presents at least one risk factor selected from the group comprising or consisting of a family history of Alzheimer's disease, Down's syndrome, a prior severe head injury, a cardiovascular disease, active smoking, obesity, diabetes, high blood pressure, and elevated cholesterol.

As used herein, “risk factor” refers to a preexisting disease, condition, habit or behavior that may lead to an increased risk of suffering from Alzheimer's disease.

In the present invention, a 2-aminoarylthiazole derivative refers to a compound characterized by the presence of a thiazolyl group substituted on position 2 (i.e., between the heterocyclic nitrogen and sulfur atoms) by a secondary or tertiary amine, wherein the nitrogen atom of the amine is substituted by at least one aryl group.

According to one embodiment, the aryl group is substituted by an arylamide group (i.e., —NH—CO-aryl).

In one embodiment, the 2-aminoarylthiazole derivative of the invention has the following formula (I):

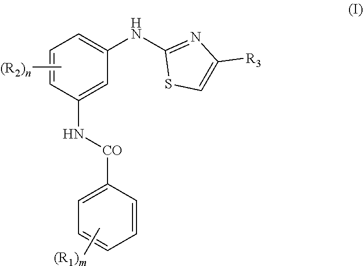

wherein:
R₁ and R₂ are selected independently from hydrogen, halogen, (C₁-C₁₀) alkyl, (C₃-C₁₀) cycloalkyl group, trifluoromethyl, alkoxy, cyano, dialkylamino, a solubilizing group, and (C₁-C₁₀) alkyl substituted by a solubilizing group;
m is 0-5;
n is 0-4;
R₃ is one of the following:
   (i) an aryl group (such as phenyl), the aryl group being optionally substituted by one or more substituents such as halogen, (C₁-C₁₀) alkyl group, trifluoromethyl, cyano and alkoxy;
   (ii) a heteroaryl group (such as 2, 3, or 4-pyridyl group), the heteroaryl group being optionally substituted by one or more substituents such as halogen, (C₁-C₁₀) alkyl group, trifluoromethyl and alkoxy;
   (iii) a five-membered ring aromatic heterocyclic group (such as, for example, 2-thienyl, 3-thienyl, 2-thiazolyl, 4-thiazolyl, 5-thiazolyl), the aromatic heterocyclic group being optionally substituted by one or more substituents such as halogen, (C₁-C₁₀) alkyl group, trifluoromethyl, and alkoxy.

Thus, in one embodiment, the 2-aminoarylthiazole derivative of the invention or a pharmaceutically acceptable salt or solvate thereof is a 2-aminoarylthiazole derivative of formula (I) or a pharmaceutically acceptable salt or solvate thereof.

In one embodiment, the 2-aminoarylthiazole derivative of the invention has the following formula (II):

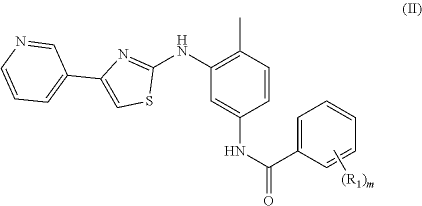

wherein:
R₁ is selected independently from hydrogen, halogen, (C₁-C₁₀) alkyl, (C₃-C₁₀) cycloalkyl group, trifluoromethyl, alkoxy, amino, alkylamino, dialkylamino, a solubilizing group, and (C₁-C₁₀) alkyl substituted by a solubilizing group; and
m is 0-5.

In one embodiment, R₁ of formula (II) is a solubilizing group. In one embodiment, R₁ of formula (II) is (C₁-C₁₀) alkyl substituted by a solubilizing group.

In one embodiment, R₁ of formula (II) is (C₁-C₁₀) alkyl-(C₂-C₁₁) heterocycloalkyl-(C₁-C₁₀) alkyl-. In one embodiment, R₁ of formula (II) is (C₁-C₄) alkyl-(C₂-C₁₁) heterocycloalkyl-(C₁-C₁₀) alkyl-, preferably (C₁-C₂) alkyl-(C₂-C₁₁) heterocycloalkyl-(C₁-C₁₀) alkyl-. In one embodiment, R₁ of formula (II) is (C₁-C₁₀) alkyl-(C₂-C₁₁) heterocycloalkyl-(C₁-C₄) alkyl-, preferably (C₁-C₁₀) alkyl-(C₂-C₁₁heterocycloalkyl-(C₁-C₂) alkyl-. In one embodiment, R₁ of formula (II) is (C₁-C₁₀) alkyl-(C₂-C₆) heterocycloalkyl-(C₁-C₁₀) alkyl-, preferably (C₁-C₁₀) alkyl-(C₄) heterocycloalkyl-(C₁-C₁₀) alkyl-. In one embodiment, R₁ of formula (II) is (C₁-C₄) alkyl-(C₂-C₆) heterocycloalkyl-(C₁-C₄) alkyl-, preferably (C₁-C₂) alkyl-(C₄) heterocycloalkyl-(C₁-C₂) alkyl-. In one embodiment, R₁ of formula (II) is (C₁-C₄) alkyl-piperazinyl-(C₁-C₄) alkyl-, preferably (C₁-C₂) alkyl-piperazinyl-(C₁-C₂) alkyl-. In one embodiment, R₁ of formula (II) is methylpiperazinyl-(C₁-C₂) alkyl-, preferably methylpiperazinyl-methyl-, more preferably 4-methylpiperazinyl-methyl-.

Thus, in one embodiment, the 2-aminoarylthiazole derivative of the invention or a pharmaceutically acceptable salt or solvate thereof is a 2-aminoarylthiazole derivative of formula (II) as described above or a pharmaceutically acceptable salt or solvate thereof.

As used herein, the term “aryl group” refers to a polyunsaturated, aromatic hydrocarbyl group having a single aromatic ring (i.e., phenyl) or multiple aromatic rings fused together (e.g., naphtyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Examples of suitable aryl groups include, without being limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C₆) aryl”.

As used herein, the term “alkyl group” refers to a saturated straight chain or branched non-cyclic hydrocarbon having from 1 to 10 carbon atoms, preferably from 1 to 6 carbon atoms. Representative saturated straight chain alkyls include, without being limited to, methyl, ethyl, n-propyl, n-butyl, n-pentyl, n-hexyl, n-heptyl, n-octyl, n-nonyl and n-decyl. Saturated branched alkyls include, without being limited to, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl. Alkyl groups included in compounds of the present invention may be optionally substituted with one or more substituents.

As used herein, the term “alkoxy” refers to an alkyl group which is attached to another moiety by an oxygen atom. Examples of alkoxy groups include, without being limited to, methoxy, isopropoxy, ethoxy, tert-butoxy. Alkoxy groups may be optionally substituted with one or more substituents.

As used herein, the term “cycloalkyl” refers to a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

As used herein, the term “halogen” refers to —F, —Cl, —Br or —I.

As used herein, the term “heteroaryl” refers to a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include, without being limited to, pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

As used herein, the term “heterocycle” refers collectively to heterocycloalkyl groups and heteroaryl groups.

As used herein, the term “heterocycloalkyl” refers to a monocyclic or polycyclic group having at least one heteroatom selected from O, N or S, and which has 2-11 carbon atoms, which may be saturated or unsaturated, but is not aromatic. Examples of heterocycloalkyl groups include, without being limited to, piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein, the term “substituent” or “substituted” means that a hydrogen radical on a compound or group is replaced with any desired group that is substantially stable to reaction conditions in an unprotected form or when protected using a protecting group. Examples of preferred substituents include, without being limited to, halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an arylalkyl, a heteroarylalkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —N—R₁₃, wherein r is 1 or 2; R₁₁ and R₁₂, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl; or R₁₁ and Rn taken together with the nitrogen to which they are attached is optionally substituted heterocycloalkyl or optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl. In certain embodiments, the term “substituent” or the adjective “substituted” refers to a solubilizing group.

As used herein, the term “solubilizing group” refers to any group which can be substantially ionized and that enables the compound to be soluble in a desired solvent, such as, for example, water or water-containing solvent (“water-solubilizing group”). Furthermore, the solubilizing group can be one that increases the compound or complex's lipophilicity. In one embodiment, the solubilizing group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. In one embodiment, the solubilizing group is one of the following:
an alkyl, cycloalkyl, aryl, heteroaryl group comprising either at least one nitrogen or oxygen heteroatom and/or which group is substituted by at least one amino group or oxo group (including, without being limited to, 2-oxopiperazinyl, 2-oxopiperidinyl, 2-oxopyrrolidinyl, 4-piperidonyl, hydantoinyl, valerolactamyl, oxiranyl, oxetanyl, tetrahydropyranyl, morpholinyl, 1,3-dioxolane, tetrahydrofuranyl and dihydrofuranyl-2-one);
an amino group which may be a saturated cyclic amino group (including, without being limited to, piperidinyl, piperazinyl and pyrrolidinyl) which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl (including, without being limited to, methyl-piperidinyl, methyl-piperazinyl and methyl-pyrrolidinyl);
one of the structures a) to i) shown below, wherein the wavy line and the arrow line correspond to the point of attachment to the core structure of the 2-aminoarylthiazole derivative of the invention, for example of formula (I) or (II):

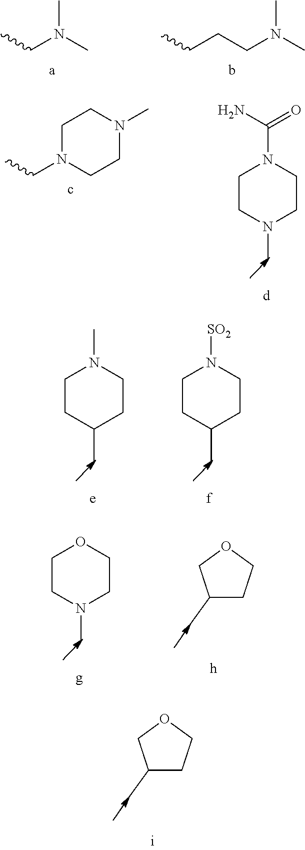

In one embodiment, the solubilizing group is one of the following:
an alkyl, cycloalkyl, aryl, heteroaryl group comprising either at least one nitrogen or oxygen heteroatom or which group is substituted by at least one amino group or oxo group;
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl;
one of the structures a) to i) shown above, wherein the wavy line and the arrow line correspond to the point of attachment to the core structure of the 2-aminoarylthiazole derivative of the invention, for example of formula (I) or (II).

In one embodiment, the solubilizing group is a saturated cyclic amino group (including, without being limited to, piperidinyl, piperazinyl and pyrrolidinyl) which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl (including, without being limited to, methyl-piperidinyl, methyl-piperazinyl and methyl-pyrrolidinyl).

In one embodiment, the solubilizing group is structure c) shown above, wherein the wavy line corresponds to the point of attachment to the core structure of the 2-aminoarylthiazole derivative of the invention, for example of formula (I) or (II).

As used herein, “pharmaceutically acceptable salt” refers to a salt of a free acid or a free base which is not biologically undesirable and is generally prepared by reacting the free base with a suitable organic or inorganic acid or by reacting the free acid with a suitable organic or inorganic base. Suitable acid addition salts are formed from acids that form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, napsylate, nicotinate, nitrate, orotate, oxalate, palmitate, pamoate, phosphate/hydrogen, phosphate/dihydrogen, phosphate, pyroglutamate, saccharate, stearate, succinate, tannate, tartrate, tosylate, trifluoroacetate and xinofoate salts. Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc salts. Hemi salts of acids and bases may also be formed, e.g., hemi sulphate and hemi calcium salts.

In one embodiment, pharmaceutically acceptable salts are pharmaceutically acceptable acid addition salts, for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid, or aromatic sulfonic acids, for example benzene-, p-toluene- or naphthalene-2-sulfonic acid.

In one embodiment, the pharmaceutically acceptable salt of the 2-aminoarylthiazole derivative of the invention is mesilate.

Unless otherwise indicated, the term “mesilate” is used herein to refer to a salt of methanesulfonic acid with a named pharmaceutical substance (such as 2-aminoarylthiazole derivatives of formula (I) or (II)). Use of mesilate rather than mesylate is in compliance with the INNM (International nonproprietary names modified) issued by WHO (e.g., World Health Organization (February 2006). International Nonproprietary Names Modified. INN Working Document 05.167/3. WHO).

As used herein, “pharmaceutically acceptable solvate” refers to a molecular complex comprising the 2-aminoarylthiazole derivative as described above and stoichiometric or sub-stoichiometric amounts of one or more pharmaceutically acceptable solvent molecules such as ethanol. The term ‘hydrate’ refers to when said solvent is water.

In one particular embodiment, the 2-aminoarylthiazole derivative as described above or a pharmaceutically acceptable salt or solvate thereof is masitinib or a pharmaceutically acceptable salt or solvate thereof.

The chemical name for masitinib is 4-(4-methylpiperazin-1-ylmethyl)-N-[4-methyl-3-(4-pyridin-3ylthiazol-2-ylamino) phenyl]benzamide-CAS number 790299-79-5:

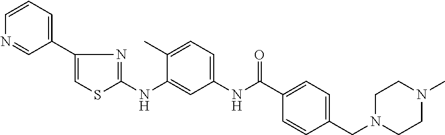 [SMILES: Cc1ccc(NC(=O)c2ccc(CN3CCN(C)CC3)cc2)cc1Nc1nc(-c2cccnc2)cs1]

Masitinib was first described in U.S. Pat. No. 7,423,055 and EP 1 525 200 Bl.

In one embodiment, the 2-aminoarylthiazole derivative of the invention or a pharmaceutically acceptable salt or solvate thereof is masitinib mesilate. Thus, in one embodiment, the pharmaceutically acceptable salt of masitinib as described above is masitinib mesilate. As mentioned above, in other words, the pharmaceutically acceptable salt of masitinib is the methanesulfonic acid salt of masitinib.

A detailed procedure for the synthesis of masitinib mesilate is given in WO 2008/098949.

In one embodiment, “masitinib mesilate” refers to the orally bioavailable mesilate salt of masitinib-CAS 1048007-93-7 (MsOH); C₂₈H₃₀N₆OS·CH₃SO₃H; MW 594.76:

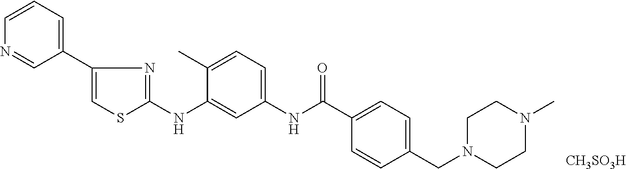

According to one embodiment, the 2-aminoarylthiazole derivative as described above, in particular masitinib, or a pharmaceutically acceptable salt or solvate thereof, is for administration at a therapeutically effective dose.

In one embodiment, the 2-aminoarylthiazole derivative as described above, in particular masitinib, or a pharmaceutically acceptable salt or solvate thereof, is for administration at a dose ranging from about 1 to about 12 mg/kg/day (mg per kilo body weight per day). In one embodiment, the 2-aminoarylthiazole derivative as described above, in particular masitinib, or a pharmaceutically acceptable salt or solvate thereof, is for administration at a dose ranging from about 1.5 to about 7.5 mg/kg/day. In one embodiment, the 2-aminoarylthiazole derivative as described above, in particular masitinib, or a pharmaceutically acceptable salt or solvate thereof, is for administration at a dose ranging from about 3 to about 12 mg/kg/day, preferably from about 3 to about 6 mg/kg/day.

In one embodiment, the 2-aminoarylthiazole derivative as described above, in particular masitinib, or a pharmaceutically acceptable salt or solvate thereof, is for administration at a dose of about 1, 2, 3, 4, 5, 6, 7, 8, 9, 10, 11, or 12 mg/kg/day. In one embodiment, the 2-aminoarylthiazole derivative as described above, in particular masitinib, or a pharmaceutically acceptable salt or solvate thereof, is for administration at a dose of about 1.5, 3, 4.5, 6, 7.5, 9, 10.5, or 12 mg/kg/day. In one embodiment, the 2-aminoarylthiazole derivative as described above, in particular masitinib, or a pharmaceutically acceptable salt or solvate thereof, is for administration at a dose of about 3, 4.5, or 6 mg/kg/day.

In one embodiment, the 2-aminoarylthiazole derivative as described above, in particular masitinib, or a pharmaceutically acceptable salt or solvate thereof, can be dose escalated by increments of about 1.5 mg/kg/day to reach a maximum of about 7.5 mg/kg/day, more preferably of about 4.5 or about 6 mg/kg/day. Each dose escalation is subjected to toxicity controls with an absence of any toxicity events permitting dose escalation to occur.

In one embodiment, the dose escalation of the 2-aminoarylthiazole derivative, in particular masitinib, or a pharmaceutically acceptable salt or solvate thereof, occurs at any time-point after at least 4 weeks after the administration of the initial dose and prior to 26 weeks after the administration of the initial dose; for example at 4 weeks, 8 weeks, 12 weeks, 16 weeks, 20 weeks, or 24 weeks after the administration of the initial dose, preferably at 12 weeks after the administration of the initial dose. Each dose escalation is subjected to toxicity controls, including for example: previous treatment period at a constant dose and no suspected severe adverse event was reported and no suspected adverse event led to treatment interruption and no suspected adverse event is ongoing at the time of the dose increase, regardless of its severity.

In one embodiment, the 2-aminoarylthiazole derivative as described above, in particular masitinib, or a pharmaceutically acceptable salt or solvate thereof, is for administration at an initial dose of about 3 mg/kg/day during 6 weeks, then at a dose of about 4.5 mg/kg/day thereafter. In one embodiment, the 2-aminoarylthiazole derivative as described above, in particular masitinib, or a pharmaceutically acceptable salt or solvate thereof, is for administration at an initial dose of about 4.5 mg/kg/day during 6 weeks, then at a dose of about 6 mg/kg/day thereafter. In one embodiment, the 2-aminoarylthiazole derivative as described above, in particular masitinib, or a pharmaceutically acceptable salt or solvate thereof, is for administration at an initial dose of about 3 mg/kg/day during 12 weeks, then at a dose of about 4.5 mg/kg/day thereafter. In one embodiment, the 2-aminoarylthiazole derivative as described above, in particular masitinib, or a pharmaceutically acceptable salt or solvate thereof, is for administration at an initial dose of about 4.5 mg/kg/day during 12 weeks, then at a dose of about 6 mg/kg/day thereafter. In one embodiment, the 2-aminoarylthiazole derivative as described above, in particular masitinib, or a pharmaceutically acceptable salt or solvate thereof, is for administration at an initial dose of about 3 mg/kg/day during at least 4 weeks, then at a dose of about 4.5 mg/kg/day during at least 4 weeks, and at a dose of about 6 mg/kg/day thereafter, with each dose escalation being subjected to toxicity controls.

According to one embodiment, any dose indicated herein refers to the amount of active ingredient as such, not to its pharmaceutically acceptable salt or solvate form. Thus, compositional variations of a pharmaceutically acceptable salt or solvate of the 2-aminoarylthiazole derivative as described above, in particular masitinib, will not impact the dose to be administered.

According to one embodiment, the 2-aminoarylthiazole derivative as described above, in particular masitinib, or pharmaceutically acceptable salt or solvate, is adapted for an administration at a dose as described above.

According to one embodiment, the 2-aminoarylthiazole derivative as described above, preferably masitinib, or a pharmaceutically acceptable salt or solvate thereof, may be administered orally, intravenously, parenterally, topically, by inhalation in particular by inhalation spray, rectally, nasally, or buccally. In one embodiment, the 2-aminoarylthiazole derivative as described above, preferably masitinib, or a pharmaceutically acceptable salt or solvate thereof, is for oral administration.

In one embodiment, the 2-aminoarylthiazole derivative as described above, preferably masitinib, or a pharmaceutically acceptable salt or solvate thereof, is for administration at least once a day, preferably twice a day. In one embodiment, the 2-aminoarylthiazole derivative as described above, preferably masitinib, or a pharmaceutically acceptable salt or solvate thereof, is for prolonged administration, such as for example, for at least 1, 2, 3, 6, 9, or 12 months.

In one embodiment, the 2-aminoarylthiazole derivative as described above, preferably masitinib, or a pharmaceutically acceptable salt or solvate thereof, is adapted or is in a form adapted for oral administration. Examples of forms adapted for oral administration include, without being limited to, liquid, paste or solid compositions, and more particularly tablets, pills, capsules, liquids, gels, syrups, slurries, and suspensions.

In one embodiment, the 2-aminoarylthiazole derivative as described above, preferably masitinib, or a pharmaceutically acceptable salt or solvate thereof, is for administration as tablets, preferably as 100 mg or 200 mg tablets.

According to one embodiment, the 2-aminoarylthiazole derivative as described above, in particular masitinib, or a pharmaceutically acceptable salt or solvate thereof, is for administration with at least one further pharmaceutically active agent.

According to the present invention, the 2-aminoarylthiazole derivative as described above, in particular masitinib, or a pharmaceutically acceptable salt or solvate thereof, may be administered simultaneously, separately, or sequentially with said at least one further pharmaceutically active agent. In one embodiment, the 2-aminoarylthiazole derivative as described above, in particular masitinib, or a pharmaceutically acceptable salt or solvate thereof, is for administration in combination with said at least one further pharmaceutically active agent, preferably in a combined preparation, pharmaceutical composition, or medicament.

Examples of further pharmaceutically active agents that may be administered to a patient with Alzheimer's disease as described herein include, without being limited to, cholinesterase inhibitors (also known as acetylcholinesterase inhibitors or Ache inhibitors) such as donepezil, rivastigmine, and galantamine; and memantine.

In one embodiment, the at least one further pharmaceutically active agent is selected from the group comprising or consisting of donepezil, rivastigmine, galantamine, and memantine.

In one embodiment, the at least one further pharmaceutically active agent is a cholinesterase inhibitor. In one embodiment, the at least one further pharmaceutically active agent is selected from the group comprising or consisting of donepezil, rivastigmine, and galantamine.

In one embodiment, the at least one further pharmaceutically active agent is memantine.

In one embodiment, the 2-aminoarylthiazole derivative as described above, in particular masitinib, or a pharmaceutically acceptable salt or solvate thereof, is for administration in combination with a cholinesterase inhibitor (in particular donepezil, rivastigmine, or galantamine) and/or with memantine.

Another object of the present invention is a method for treating Alzheimer's disease in a patient in need thereof as defined above, comprising administering to the patient a 2-aminoarylthiazole derivative, in particular masitinib, or a pharmaceutically acceptable salt or solvate thereof, as described above.

In one embodiment, the present invention relates to a method for treating less severe and/or early-stage Alzheimer's disease as defined above, in a patient in need thereof, in particular in a patient in need thereof as defined above, comprising administering to the patient a 2-aminoarylthiazole derivative, in particular masitinib, or a pharmaceutically acceptable salt or solvate thereof, as described above.

In one embodiment, the present invention relates to a method for treating early-stage Alzheimer's dementia as defined above, in a patient in need thereof, in particular in a patient in need thereof as defined above, comprising administering to the patient a 2-aminoarylthiazole derivative, in particular masitinib, or a pharmaceutically acceptable salt or solvate thereof, as described above.

In one embodiment, the present invention relates to a method for treating Alzheimer's disease in a patient in need thereof, said patient having a baseline ADAS-Cog score equal to or lower than 40, having a baseline ADCS-ADL score equal to or greater, preferably greater, than 32, having a baseline MMSE score equal to or greater than 13, and/or having a time from diagnosis to treatment initiation with masitinib, or a pharmaceutically acceptable salt or solvate thereof, equal to or less than 5 years, the method comprising administering to the patient a 2-aminoarylthiazole derivative, in particular masitinib, or a pharmaceutically acceptable salt or solvate thereof, as described above.

In one embodiment, the method as described above comprises administering at least one further pharmaceutically active agent as described above.

Another object of the present invention is a pharmaceutical composition for use in the treatment of Alzheimer's disease in a patient in need thereof as defined above, said pharmaceutical composition comprising a 2-aminoarylthiazole derivative, in particular masitinib, or a pharmaceutically acceptable salt or solvate thereof, as described above and optionally at least one pharmaceutically acceptable excipient.

Another object of the present invention is a pharmaceutical composition for the treatment of Alzheimer's disease in a patient in need thereof as defined above, said pharmaceutical composition comprising a 2-aminoarylthiazole derivative, in particular masitinib, or a pharmaceutically acceptable salt or solvate thereof, as described above and optionally at least one pharmaceutically acceptable excipient.

In one embodiment, the pharmaceutical composition of the invention is for use in the treatment of less severe and/or early-stage Alzheimer's disease as defined above. In one embodiment, the pharmaceutical composition of the invention is for use in the treatment of early-stage Alzheimer's dementia as defined above.

In one embodiment, the pharmaceutical composition of the invention is for use in combination with at least one further pharmaceutically active agent as described above.

Another object of the present invention is the use of a 2-aminoarylthiazole derivative, in particular masitinib, or a pharmaceutically acceptable salt or solvate thereof, as described above, for the manufacture of a medicament for the treatment of Alzheimer's disease in a patient in need thereof as defined above.

In one embodiment, the medicament is for the treatment of less severe and/or early-stage Alzheimer's disease as defined above. In one embodiment, the medicament is for the treatment of early-stage Alzheimer's dementia as defined above.

In one embodiment, the medicament is for use in combination with at least one further pharmaceutically active agent as described above.

### EXAMPLES

The present invention is further illustrated by the following examples.

### Example 1: Study AB09004 Design

An international, multicenter, randomized, double-blind, placebo-controlled phase 3 study was conducted with the objective to evaluate oral masitinib at a dose of 4.5 mg/kg/day as a treatment for Alzheimer's disease.

Eligible patients, aged at least 50 years, were diagnosed with dementia of Alzheimer's type according to Diagnostic and Statistical Manual of Mental Disorders (DSM)-IV criteria, and probable Alzheimer's disease according to NINCDS-ADRDA (National Institute of Neurological and Communicative Diseases and Stroke/Alzheimer's Disease and Related Disorders Association) criteria. Patients had a baseline score on the Mini-Mental State Examination (MMSE) ranging from 12 to 25. Patients with any other cause of dementia not due to Alzheimer's disease were ineligible for the study.

Eligible patients were treated during 24 weeks with masitinib at a dose of 4.5 mg/kg/day (mg per kilo body weight per day) or with placebo. Treatment was administered as add-on therapy in patients who had been treated for a minimum of 6 months with a stable dose of cholinesterase inhibitors (donepezil, rivastigmine or galantamine) and/or memantine, with no changes foreseen in therapy throughout the study.

Primary analysis was based on the co-primary endpoints of:
Change from baseline in Alzheimer's Disease Cooperative Study Activities of Daily Living (ADCS-ADL) score. The ADCS-ADL assesses the competence of patients with Alzheimer's disease in basic and instrumental activities of daily living (ADLs).
Change from baseline in Alzheimer's Disease Assessment Scale-Cognitive Subscale (ADAS-Cog). The ADAS-Cog assesses cognition and memory.

The treatment effect needs to be established in at least one of these endpoints and both endpoints will be simultaneously tested at a 2.5% level of significance (p<0.025) with a fallback procedure.

Treatment effect (masitinib versus placebo) was assessed based on absolute change from baseline to week 24 by using an analysis of covariance model (ANCOVA linear model) with the following factors: treatment, baseline value, and stratification criteria of baseline MMSE score, age, ADCS-ADL total score, and ADAS-Cog total score.

For ADCS-ADL, the global score (i.e., the sum of 23 items rated by the caregiver) was used with a range from 0 to 78, wherein 78 implies full functioning with no impairment. Thus, a higher score signifies greater functional ability, while a lower score signifies greater (worse) impairment. Results are expressed as a least-squares means difference (δLSM) of absolute change in ADCS-ADL score from baseline; that is to say, the change over time in the ADCS-ADL score, or in other words the difference between the ADCS-ADL score at baseline (before treatment initiation) and the ADCS-ADL score after treatment with masitinib or with placebo. An increase in ADCS-ADL score with respect to baseline (i.e., a positive δLSM) indicates an improvement in the patient and therefore disease stability, whereas a decrease in ADCS-ADL score with respect to baseline (i.e., negative δLSM) indicates a worsening in the patient and therefore disease progression. Treatment-effect is reported as the difference between treatment-arms (ΔLSM) (calculated as δLSM[masitinib] minus δLSM[placebo]), with a positive value indicating a beneficial effect of treatment with masitinib as compared to treatment with placebo. In a scenario where both treatment-arms show worsening in activities of daily living (i.e., a negative δLSM), then a positive ΔLSM indicates a beneficial treatment effect for masitinib, i.e., the rate of decline in masitinib treated patients is slower than that of patients from the placebo-arm.

The ADAS-Cog version used in study AB09004 included 11 items regrouped as follows: language (items 1, 2, 3), praxia and gnosia (items 5, 7, 8, 9), memory (items 4, 10, 11), orientation (item 6). The items range either from 0 to 5 (items 1, 2, 3, 5, 7, 8, 9, 11), or from 0 to 8 (item 6), or from 0 to 10 (item 4), or from 0 to 12 (item 10). The global score, which is the sum of the 11 items, ranges from 0 to 70, with higher scores indicating greater cognitive impairment. Results are expressed as a least-squares means difference (δLSM) of absolute change in ADAS-Cog score from baseline; that is to say, the change over time in the ADAS-Cog score, or in other words the difference between the ADAS-Cog score at baseline and the ADAS-Cog score after treatment with masitinib or with placebo. A decrease in ADAS-Cog score with respect to baseline (i.e., a negative δLSM) indicates an improvement in the patient and therefore disease stability, whereas an increase in ADAS-Cog score with respect to baseline (i.e., a positive δLSM) indicates a worsening in the patient and therefore disease progression. Treatment-effect is reported as the difference between treatment-arms (ΔLSM) (calculated as δLSM[masitinib] minus δLSM[placebo]), with a negative value indicating a beneficial effect of treatment with masitinib as compared with placebo. In a scenario where both treatment-arms show worsening in cognitive function (i.e., a positive δLSM), then a negative ΔLSM indicates a beneficial treatment effect for masitinib, i.e., the rate of decline in masitinib treated patients is slower than that of patients from the placebo-arm.

### Example 2: Analysis of Subpopulations According to the Clinical Markers of ‘Time from Diagnosis Until Treatment Initiation’, ‘Baseline ADAS-Cog Score’, ‘Baseline ADCS-ADL Score’, and ‘Baseline MMSE Score’

### Methods

Alzheimer's disease may be described as a biological continuum that includes the hallmark pathological processes of amyloid-beta dysmetabolism, formation of amyloid deposits and neurofibrillary tangles, and neurodegeneration. Without wishing to be bound by any theory, the Inventors suggest that masitinib treatment-effect will correlate with pathophysiological influence of the drug's targeted mechanism; for example, dynamic processes such as innate immunity-related inflammation within the central nervous system (CNS) that will have differential effects along the disease trajectory. Hence, by targeting a treatment-susceptible phase of Alzheimer's disease, for example via modulation or priming of macrophage/microglia and mast cells, it may be possible to inhibit neurological damage and promote CNS repair pathways. Exactly how such differential CNS innate immunity-related processes contribute to the temporal progression sequence of Alzheimer's disease pathogenesis remains unknown.

In the absence of established prognostic or predictive biological markers that identify which patients with Alzheimer's disease are most likely to benefit from masitinib treatment, it is necessary to use accessible clinical markers. The clinical markers of: ‘time from diagnosis’ (at baseline or treatment initiation); Alzheimer's Disease Assessment Scale—Cognitive Subscale (ADAS-Cog) score of a given threshold (at baseline or treatment initiation); Alzheimer's Disease Cooperative Study—Activities of Daily Living (ADCS-ADL) score of a given threshold (at baseline or treatment initiation); and/or Mini-Mental State Examination (MMSE) score of a given range (at baseline or treatment initiation), have potential predictive value in identifying the patients most likely to benefit from masitinib treatment, and in showing whether it is beneficial to treat at an earlier phase of the neurodegenerative disease process (i.e., in patients prior to advanced neurological damage) or at a later phase of the neurodegenerative disease process (i.e., in patients with preexisting advanced neurological damage).

‘Time from diagnosis’ of Alzheimer's disease, is ideally based on a clinically definite diagnosis of Alzheimer's disease as determined by an Alzheimer's disease specialist neurologist. In practice it is derived from the date (year/month/day, year/month, or year) of first clinically definite diagnosis of Alzheimer's disease, i.e., according to the DSM-IV and NINCDS-ADRDA criteria, with duration calculated from date of treatment initiation.

Subpopulation analyses of the co-primary endpoint data from study AB09004 assessed the impact of the above-listed clinical markers.

Results surprisingly show that only those patients receiving treatment at an earlier phase of the neurodegenerative disease process and/or prior to onset of a certain threshold of functional impairment are susceptible to benefit from masitinib treatment. Patients in a more advanced phase of Alzheimer's disease development do not display a discernable response to masitinib as compared with placebo. Hence, the aforementioned clinical markers can be used as predictive indicators of likely response to masitinib and therefore as an instrument for selection of patients most likely to benefit from masitinib treatment; for example, patients with Alzheimer's disease having an ADAS-Cog score lower than or equal to 40, an ADCS-ADL score equal to or greater than 32, a MMSE score equal to or greater than 13, preferably equal to or greater than 14, and/or a time from diagnosis to treatment initiation with masitinib of less than or equal to 5 years, preferably equal to or less than 3 years.

### Results

More specifically, the abovementioned clinical data show that orally administered masitinib (4.5 mg/kg/day) provides greater therapeutic benefit to a distinct subpopulation of the Alzheimer's disease population (see Tables 1-6 below). This enhanced treatment-effect, as measured by change in either the ADAS-Cog score or the ADCS-ADL score, is identified in subpopulations that are defined in terms of time from diagnosis, level of cognitive deficit, or level of impaired functioning in daily life.

In the absence of established prognostic or predictive biological markers that identify which Alzheimer's disease patients are most likely to benefit from masitinib treatment, it is necessary to use accessible clinical markers. The marker ‘time from diagnosis’ (at baseline or treatment initiation) has potential predictive value in showing whether it is beneficial to treat at a certain point of the Alzheimer's disease continuum (i.e., at an earlier or later phase of the disease process). Results showed that subpopulations of Alzheimer's disease patients defined as having time from diagnosis to treatment initiation less than or equal to the cut-offs of 5 years, 4 years, 3 years, or 2 years, demonstrated a statistically significant difference (p<0.025) in change of ADAS-Cog from baseline following masitinib treatment as compared with placebo. This treatment effect was not evident for the associated complementary subpopulations (i.e., time from diagnosis to treatment initiation greater than 5 years, 4 years, 3 years or 2 years).

For example, the subpopulation of Alzheimer's disease patients defined as having ‘time from diagnosis to treatment initiation of less than or equal to 3 years’ demonstrated a statistically significant difference (p<0.025) in change of ADAS-Cog from baseline following masitinib treatment as compared with placebo. The difference between treatment-arms (ΔLSM), was −2.8700 points in favor of masitinib (p=0.00004) (see Table 1 below). In contrast, the complementary subpopulation defined as having ‘time from diagnosis to treatment initiation of greater than 3 years’ showed no significant masitinib treatment-effect with respect to placebo (ΔLSM of −0.3770 points, p=0.74309).

For example, the subpopulation of Alzheimer's disease patients defined as having ‘time from diagnosis to treatment initiation of less than or equal to 2 years’ demonstrated a statistically significant difference (p<0.025) in change of ADAS-Cog from baseline following masitinib treatment as compared with placebo. The difference between treatment-arms (ΔLSM), was −2.7754 points in favor of masitinib (p=0.00009) (see Table 1 below). In contrast, the complementary subpopulation defined as having ‘time from diagnosis to treatment initiation of greater than 2 years’ showed no significant masitinib treatment-effect with respect to placebo (ΔLSM of −1.3581 points, p=0.112).

TABLE 1
Comparison of masitinib- versus placebo-treated patients in
subpopulations defined using the clinical marker of time from diagnosis
to treatment initiation (according to ADAS-Cog assessment)

| | | | δLSM | ΔLSM | |
|---|---|---|---|---|---|
| | | | (ADAS- | (ADAS- | |
| Subpopulation | Arm | N | Cog) | Cog) | p-value |
| Diagnosis ≤2 | M4.5 | 95 | −1.6958 | −2.7754 | 0.0009 |
| years | PBO | 96 | 1.0796 | | |
| Diagnosis >2 | M4.5 | 87 | −1.2197 | −1.3581 | 0.112 (NS) |
| years | PBO | 80 | 0.1385 | | |
| Diagnosis ≤3 | M4.5 | 127 | −1.7671 | −2.8700 | 0.00004 |
| years | PBO | 133 | 1.1029 | | |
| Diagnosis >3 | M4.5 | 55 | −0.7048 | −0.3770 | 0.74309 (NS) |
| years | PBO | 43 | −0.3277 | | |
| Diagnosis ≤4 | M4.5 | 150 | −1.4948 | −2.3435 | 0.00045 |
| years | PBO | 155 | 0.8487 | | |
| Diagnosis >4 | M4.5 | 32 | −1.2607 | −1.3426 | 0.31130 (NS) |
| years | PBO | 21 | 0.0819 | | |
| Diagnosis ≤5 | M4.5 | 160 | −1.4394 | −2.2599 | 0.00043 |
| years | PBO | 161 | 0.8205 | | |
| Diagnosis >5 | M4.5 | 22 | −1.4756 | −1.4470 | 0.42256 (NS) |
| years | PBO | 15 | −0.0286 | | |

N: number of patients in subpopulation. ΔLSM: Difference between treatment-arms (treatment-effect) calculated as δLSM[masitinib] minus δLSM[placebo]. δLSM: least-squares means difference of absolute change in ADAS-Cog score at week 24 from baseline. Negative ΔLSM (ADAS-Cog) indicates a greater baseline difference for masitinib as compared with placebo. Statistically significant difference between treatment-arms is defined as p-value of <0.025.

The variable of baseline MMSE (or MMSE before or at time of treatment initiation) has potential predictive value in showing whether it is beneficial to treat at a certain point of the Alzheimer's disease continuum. Results showed that subpopulations of Alzheimer's disease patients defined as having baseline MMSE of greater than or equal to the cut-offs of 13, 14, 15, 16, or 17, demonstrated a statistically significant difference (p<0.025) in change of ADAS-Cog from baseline or change of ADCS-ADL from baseline following masitinib treatment as compared with placebo. This treatment effect was not evident for the associated complementary subpopulations (i.e., baseline MMSE lower than 13, 14, 15, 16, or 17).

For example, the subpopulation of Alzheimer's disease defined by ‘baseline MMSE score greater than or equal to 14 points before treatment initiation’ showed a significant masitinib treatment-effect with respect to placebo in terms of change of ADCS-ADL from baseline (ΔLSM=+2.4046, p=0.00875) and also in terms of change of ADAS-Cog from baseline (ΔLSM=−2.4192, p=0.00012) (see Table 2 below). In contrast, the complementary subpopulation defined as having ‘baseline MMSE less than 14 points before treatment initiation’ showed no significant masitinib treatment-effect with respect to placebo in terms of change of ADCS-ADL from baseline (ΔLSM of −3.5751 points, p=0.26527) or in terms of change of ADAS-Cog from baseline (ΔLSM=1.0497, p=0.56013).

TABLE 2
Comparison of masitinib- versus placebo-treated patients in
subpopulations defined using the clinical marker of baseline MMSE score
(according to ADCS-ADL and ADAS-Cog assessments)

| | | | ADCS-ADL | | | ADAS-Cog | | |
|---|---|---|---|---|---|---|---|---|
| Subpop | Arm | N | δLSM | ΔLSM | p-value | δLSM | ΔLSM | p-value |
| MMSE ≥13 | M4.5 | 179 | 1.1204 | 2.0496 | 0.02183 | −1.4717 | −2.1488 | 0.00044 |
| | PBO | 170 | −0.9292 | | | 0.6771 | | |
| MMSE <13 | M4.5 | 3 | −10.9758 | −9.2849 | 0.06036 (NS) | 2.1702 | −0.4996 | 0.83027 (NS) |
| | PBO | 6 | −1.6909 | | | 2.6698 | | |
| MMSE ≥14 | M4.5 | 164 | 1.3505 | 2.4046 | 0.00875 | −1.8028 | −2.4192 | 0.00012 |
| | PBO | 156 | −1.0541 | | | 0.6164 | | |
| MMSE <14 | M4.5 | 18 | −4.6507 | −3.5751 | 0.26527 (NS) | 3.6165 | 1.0497 | 0.56013 (NS) |
| | PBO | 20 | −1.0756 | | | 2.5669 | | |
| MMSE ≥15 | M4.5 | 151 | 1.3520 | 2.2738 | 0.01945 | −1.8834 | −2.2577 | 0.00049 |
| | PBO | 140 | −0.9218 | | | 0.3742 | | |
| MMSE <15 | M4.5 | 31 | −3.1413 | −0.5657 | 0.79090 (NS) | 1.5482 | −1.0976 | 0.45653 (NS) |
| | PBO | 36 | −2.5756 | | | 2.6458 | | |
| MMSE ≥16 | M4.5 | 140 | 1.6088 | 2.5566 | 0.01163 | −2.0435 | −2.2907 | 0.00053 |
| | PBO | 130 | −0.9478 | | | 0.2473 | | |
| MMSE <16 | M4.5 | 42 | −2.3199 | −0.6926 | 0.69930 (NS) | 0.8538 | −1.0624 | 0.40095 (NS) |
| | PBO | 46 | −1.6272 | | | 1.9162 | | |
| MMSE ≥17 | M4.5 | 128 | 1.0330 | 2.2870 | 0.02269 | −2.3004 | −2.4915 | 0.00024 |
| | PBO | 123 | −1.2540 | | | 0.1911 | | |
| MMSE <17 | M4.5 | 54 | −1.0314 | 0.5605 | 0.75097 (NS) | 0.9251 | −1.0127 | 0.37421 (NS) |
| | PBO | 53 | −1.5920 | | | 1.9378 | | |

N: number of patients in subpopulation.
ΔLSM: Difference between treatment-arms (treatment-effect) calculated as δLSM[masitinib] minus δLSM[placebo].
δLSM: least-squares means difference of absolute change in ADCS-ADL score at week 24 from baseline.
Positive ΔLSM (ADCS-ADL) indicates a greater baseline difference for masitinib as compared with placebo.
Negative ΔLSM (ADAS-Cog) indicates a greater baseline difference for masitinib as compared with placebo.
Statistically significant difference between treatment-arms is defined as p-value of <0.025.

The subpopulation of Alzheimer's disease defined by ‘baseline MMSE score ranging from 25 to 21 points before treatment initiation’ showed a significant masitinib treatment-effect with respect to placebo in terms of change of ADCS-ADL from baseline (ΔLSM=+2.7435, p=0.024) and also in terms of change of ADAS-Cog from baseline (ΔLSM=−2.8929, p=0.0008) (see Table 3 below). In contrast, the complementary subpopulation defined as having ‘baseline MMSE score ranging from 20 to 12 points before treatment initiation’ showed no significant masitinib treatment-effect with respect to placebo in terms of change of ADCS-ADL from baseline (ΔLSM of +1.3770 points, p=0.245) or in terms of change of ADAS-Cog from baseline (ΔLSM=−1.7387, p=0.0284). This corresponds to an improved benefit of between 99% to 66% in the subpopulation ‘baseline MMSE score ranging from 25 to 21 points before treatment initiation’ relative to the subpopulation ‘baseline MMSE score ranging from 20 to 12 points before treatment initiation’.

TABLE 3
Comparison of masitinib- versus placebo-treated patients in
subpopulations defined using the clinical marker of baseline MMSE score
(according to ADCS-ADL and ADAS-Cog assessments)

| | | | ADCS-ADL | | | ADAS-Cog | | |
|---|---|---|---|---|---|---|---|---|
| Subpop | Arm | N | δLSM | ΔLSM | p-value | δLSM | ΔLSM | p-value |
| MMSE [25-21] | M4.5 | 63 | 3.5738 | 2.7435 | 0.024 | −2.4719 | −2.8929 | 0.0008 |
| | PBO | 61 | 0.8302 | | | 0.4210 | | |
| MMSE [12-20] | M4.5 | 119 | −0.4369 | 1.3770 | 0.245 (NS) | −1.0350 | −1.7387 | 0.0284 (NS) |
| | PBO | 115 | −1.8139 | | | 0.7037 | | |

N: number of patients in subpopulation.
ΔLSM: Difference between treatment-arms (treatment-effect) calculated as δLSM[masitinib] minus δLSM[placebo].
δLSM: least-squares means difference of absolute change in ADCS-ADL score at week 24 from baseline.
Positive ΔLSM (ADCS-ADL) indicates a greater baseline difference for masitinib as compared with placebo.
Negative ΔLSM (ADAS-Cog) indicates a greater baseline difference for masitinib as compared with placebo.
Statistically significant difference between treatment-arms is defined as p-value of <0.025.

The variable of baseline ADAS-Cog score (or ADAS-Cog score before or at time of treatment initiation) has potential predictive value in showing whether it is beneficial to treat at a certain point of the Alzheimer's disease continuum. Results showed that subpopulations of Alzheimer's disease patients defined as having baseline ADAS-Cog of less than or equal to the cut-offs of 25, 32, 35 or 40, demonstrated a statistically significant difference (p<0.025) in change of ADAS-Cog from baseline, or change of ADCS-ADL from baseline, following masitinib treatment as compared with placebo. This treatment effect was not evident for the associated complementary subpopulations (i.e., baseline ADAS-Cog of greater than 25, 32, 35 or 40).

For example, the subpopulation of Alzheimer's disease patients defined by an ‘ADAS-Cog score less than or equal to 25 points before treatment initiation’ showed a trend masitinib treatment-effect with respect to placebo in terms of change of ADCS-ADL from baseline (ΔLSM=+2.3646, p=0.0262) (see Table 4 below). In contrast, the complementary subpopulation defined as having ‘ADAS-Cog score greater than 25 points before treatment initiation’ showed no significant masitinib treatment-effect with respect to placebo (ΔLSM of +1.2592 points, p=0.367). This corresponds to an improved benefit of 84% in the subpopulation ‘ADAS-Cog score less than or equal to 25 points before treatment initiation’ relative to the subpopulation ‘ADAS-Cog score greater than 25 points before treatment initiation’.

For example, the subpopulation of Alzheimer's disease defined by an ‘ADAS-Cog score less than or equal to 35 points before treatment initiation’ showed a significant masitinib treatment-effect with respect to placebo in terms of change of ADCS-ADL from baseline (ΔLSM=+2.2189, p=0.0216) (see Table 4 below). In contrast, the complementary subpopulation defined as having ‘ADAS-Cog score greater than 35 points before treatment initiation’ showed no discernable treatment-effect with respect to placebo (ΔLSM of −0.788 points, p=0.7203).

TABLE 4
Comparison of masitinib- versus placebo-treated
patients in subpopulations
defined using the clinical marker of baseline ADAS-Cog score

| | | ΔLSM | |
|---|---|---|---|
| Subpopulation | N | (ADCS-ADL) | p-value |
| Baseline ADAS-Cog | 180 | 2.3646 | 0.0262 (NS) |
| ≤25 | | | |
| Baseline ADAS-Cog | 178 | 1.2592 | 0.367 (NS) |
| >25 | | | |
| Baseline ADAS-Cog | 290 | 2.2189 | 0.0216 |
| ≤35 | | | |
| Baseline ADAS-Cog | 68 | −0.788 | 0.7203 (NS) |
| >35 | | | |

N: number of patients in subpopulation. ΔLSM: Difference between treatment-arms (treatment-effect) calculated as δLSM[masitinib] minus δLSM[placebo]. δLSM: least-squares means difference of absolute change in ADCS-ADL score at week 24 from baseline. Positive ΔLSM (ADCS-ADL) indicates a greater baseline difference for masitinib as compared with placebo. Statistically significant difference between treatment-arms is defined as p-value of <0.025.

For example, the subpopulation of Alzheimer's disease patients defined by an ‘ADAS-Cog score less than or equal to 40 points before treatment initiation’ showed a trend masitinib treatment-effect with respect to placebo in terms of change of ADAS-Cog from baseline (ΔLSM=−2.3299, p=0.00020) (see Table 5 below). In contrast, the complementary subpopulation defined as having ‘ADAS-Cog score greater than 40 points before treatment initiation’ showed no significant masitinib treatment-effect with respect to placebo (ΔLSM of −0.2351 points, p=0.91116).

TABLE 5
Comparison of masitinib- versus placebo-treated patients in
subpopulations defined using the clinical marker of baseline
ADAS-Cog score (according to ADAS-Cog assessment)

| | | | 8LSM | ALSM | |
|---|---|---|---|---|---|
| Subpopulation | Arm | N | (ADAS-Cog) | (ADAS-Cog) | p-value |
| Baseline | M4.5 | 164 | −1.4472 | −2.3299 | 0.00020 |
| ADAS-Cog ≤40 | PBO | 158 | 0.8827 | | |
| Baseline | M4.5 | 18 | −4.9348 | −0.2351 | 0.91116 |
| ADAS-Cog >40 | PBO | 18 | −4.6997 | | (NS) |
| Baseline | M4.5 | 137 | −1.5266 | −2.1011 | 0.00140 |
| ADAS-Cog ≤32 | PBO | 128 | 0.5744 | | |
| Baseline | M4.5 | 45 | −7.2739 | −1.6044 | 0.22797 |
| ADAS-Cog >32 | PBO | 48 | −5.6694 | | (NS) |

N: number of patients in subpopulation. ΔLSM: Difference between treatment-arms (treatment-effect) calculated as δLSM[masitinib] minus δLSM[placebo]. δLSM: least-squares means difference of absolute change in ADCS-ADL score at week 24 from baseline. Negative ΔLSM (ADAS-Cog) indicates a greater baseline difference for masitinib as compared with placebo. Statistically significant difference between treatment-arms is defined as p-value of <0.025.

The variable of baseline ADCS-ADL score (or ADCS-ADL score before or at time of treatment initiation) has potential predictive value in showing whether it is beneficial to treat at a certain point of the Alzheimer's disease continuum. Results showed that subpopulations of Alzheimer's disease patients defined as having baseline ADCS-ADL of greater than or equal to the cut-offs of 32, 35, 38, 41, 47, or 50, or having baseline ADCS-ADL of greater than the cut-offs of 39 and 55, demonstrated a statistically significant difference (p<0.025) or trend difference in change of ADCS-ADL from baseline following masitinib treatment as compared with placebo. This treatment effect was not evident for the associated complementary subpopulations.

For example, the subpopulation of Alzheimer's disease patients defined by an ‘ADCS-ADL score greater than 39 points before treatment initiation’ showed a trend masitinib treatment-effect with respect to placebo in terms of change of ADCS-ADL from baseline (ΔLSM=+2.0733, p=0.0433) (see Table 6 below). In contrast, the complementary subpopulation defined as having ‘ADCS-ADL score less than or equal to 39 points before treatment initiation’ showed no significant masitinib treatment-effect with respect to placebo (ΔLSM of +0.7132 points, p=0.686). This corresponds to an improved benefit of 191% in the subpopulation ‘ADCS-ADL score greater than 39 points before treatment initiation’ relative to the subpopulation ‘ADCS-ADL score less than or equal to 39 points before treatment initiation’.

For example, subpopulation of Alzheimer's disease defined by an ‘ADCS-ADL score greater than 55 points before treatment initiation’ showed a significant masitinib treatment-effect with respect to placebo in terms of change of ADCS-ADL from baseline (ΔLSM=+3.1871, p=0.0077) (see Table 6 below). In contrast, the complementary subpopulation defined as having ‘ADCS-ADL score less than or equal to 55 points before treatment initiation’ showed no significant masitinib treatment-effect with respect to placebo (ΔLSM of +0.4577 points, p=0.727). This corresponds to an improved benefit of about 600% in the subpopulation ‘ADCS-ADL score greater than 55 points before treatment initiation’ relative to the subpopulation ‘ADCS-ADL score less than or equal to 55 points before treatment initiation’.

For example, the subpopulation of Alzheimer's disease defined by an ‘ADCS-ADL score greater than or equal to 50 points before treatment initiation’ showed a significant masitinib treatment-effect with respect to placebo in terms of change of ADCS-ADL from baseline (ΔLSM=+3.2315, p=0.00704) (see Table 6 below). In contrast, the complementary subpopulation defined as having ‘ADCS-ADL score less than 50 points before treatment initiation’ showed no significant masitinib treatment-effect with respect to placebo (ΔLSM of +0.3062 points, p=0.81375).

For example, the subpopulation of Alzheimer's disease defined by an ‘ADCS-ADL score greater than or equal to 38 points before treatment initiation’ showed a significant masitinib treatment-effect with respect to placebo in terms of change of ADCS-ADL from baseline (ΔLSM=+2.3285, p=0.01996) (see Table 6 below). In contrast, the complementary subpopulation defined as having ‘ADCS-ADL score less than 38 points before treatment initiation’ showed no significant masitinib treatment-effect with respect to placebo (ΔLSM of −0.2109 points, p=0.91442).

TABLE 6
Comparison of masitinib- versus placebo-treated patients in
subpopulations defined using the clinical marker of baseline
ADCS-ADL score (according to ADCS-ADL assessment)

| | | | δLSM | ΔLSM | |
|---|---|---|---|---|---|
| | | | (ADCS- | (ADCS- | |
| Subpopulation | Arm | N | ADL) | ADL) | p-value |
| Baseline ADCS- | M4.5 | 162 | 0.9536 | 1.9799 | 0.03495 |
| ADL ≥32 | PBO | 158 | −1.0263 | | |
| Baseline ADCS- | M4.5 | 20 | 0.8656 | −0.9123 | 0.72094 |
| ADL <32 | PBO | 18 | 1.7778 | | (NS) |
| Baseline ADCS- | M4.5 | 151 | 0.9909 | 2.0209 | 0.03711 |
| ADL ≥35 | PBO | 149 | −1.0300 | | |
| Baseline ADCS- | M4.5 | 31 | 0.9816 | 0.2942 | 0.89141 |
| ADL <35 | PBO | 27 | 0.6874 | | (NS) |
| Baseline ADCS- | M4.5 | 146 | 1.0071 | 2.3285 | 0.01996 |
| ADL ≥38 | PBO | 138 | −1.3214 | | |
| Baseline ADCS- | M4.5 | 36 | 1.9901 | −0.2109 | 0.91442 |
| ADL <38 | PBO | 38 | 2.2010 | | (NS) |
| Baseline ADCS- | M4.5 | 138 | 0.6655 | 2.0733 | 0.0433 |
| ADL >39 | PBO | 134 | −1.4078 | | |
| Baseline ADCS- | M4.5 | 44 | 2.8513 | 0.7132 | 0.686 |
| ADL ≤39 | PBO | 42 | 2.1381 | | (NS) |
| Baseline ADCS- | M4.5 | 134 | 0.8077 | 2.0192 | 0.05122 |
| ADL ≥41 | PBO | 133 | −1.2114 | | |
| Baseline ADCS- | M4.5 | 48 | 3.2426 | 1.0592 | 0.52964 |
| ADL <41 | PBO | 43 | 2.1834 | | (NS) |
| Baseline ADCS- | M4.5 | 125 | 1.0869 | 2.1676 | 0.05177 |
| ADL ≥47 | PBO | 117 | −1.0806 | | |
| Baseline ADCS- | M4.5 | 57 | 2.0776 | 0.9542 | 0.50515 |
| ADL <47 | PBO | 59 | 1.1234 | | (NS) |
| Baseline ADCS- | M4.5 | 93 | 0.7638 | 3.2315 | 0.00704 |
| ADL ≥50 | PBO | 85 | −2.4677 | | |
| Baseline ADCS- | M4.5 | 89 | 1.5300 | 0.3062 | 0.81375 |
| ADL <50 | PBO | 91 | 1.2238 | | (NS) |
| Baseline ADCS- | M4.5 | 89 | 0.8306 | 3.1871 | 0.0077 |
| ADL >55 | PBO | 81 | −2.3565 | | |
| Baseline ADCS- | M4.5 | 93 | 1.3484 | 0.4577 | 0.727 |
| ADL ≤55 | PBO | 95 | 0.8907 | | (NS) |

N: number of patients in subpopulation. ΔLSM: Difference between treatment-arms (treatment-effect) calculated as δLSM[masitinib] minus δLSM[placebo]. δLSM: least-squares means difference of absolute change in ADCS-ADL score at week 24 from baseline. Positive ΔLSM (ADCS-ADL) indicates a greater baseline difference for masitinib as compared with placebo. Statistically significant difference between treatment-arms is defined as p-value of <0.025.

### Example 3: Comparison of Treatment-Effect Observed in the Overall Study Population Versus in Subpopulations According to the Clinical Markers of ‘Time from Diagnosis Until Treatment Initiation’, ‘Baseline MMSE Score’, and ‘Baseline ADCS-ADL Score’

As detailed above, masitinib (4.5 mg/kg/day) provides an increased clinical benefit in the subpopulations of Alzheimer's disease patients defined according to the clinical markers of ‘time from diagnosis until treatment initiation’, ‘baseline MMSE score’, ‘baseline ADAS-Cog score’, and ‘baseline ADCS-ADL score’. Said increased clinical benefit is notably observed as a substantial treatment-effect (expressed in terms of a statistically significant difference between treatment-arms) in the subpopulations of Alzheimer's disease patients as defined herein, as compared to the associated complementary subpopulations (see Tables 1-6 above).

The treatment-effect (expressed in terms of a statistically significant difference between treatment-arms) observed in the subpopulations of Alzheimer's disease patients as defined herein was also compared to the treatment-effect (expressed in terms of a statistically significant difference between treatment-arms) observed in the overall study population.

The comparison was carried out using the following information:
δLSM: least-squares means difference of absolute change in endpoint score at week 24 from baseline, either according to ADAS-Cog assessment or to ADCS-ADL assessment.
ΔLSM: Difference between treatment-arms (treatment-effect) calculated as δLSM[masitinib] minus δLSM[placebo].
Δsub/Δoverall: Ratio of subpopulation's placebo-controlled treatment-effect (ΔLSM at W24 for masitinib 4.5 mg/kg/d parallel arm) versus overall study population's placebo-controlled treatment-effect (ΔLSM at W24 for masitinib 4.5 mg/kg/d parallel arm). A ratio >1.0 indicates an improved placebo-controlled treatment-effect (expressed in percentage improvement) in the subpopulation as compared to the overall study population.
Negative ΔLSM (ADAS-Cog) indicates a greater baseline difference for masitinib as compared with placebo.
Positive ΔLSM (ADCS-ADL) indicates a greater baseline difference for masitinib as compared with placebo.

As illustrated in Table 7-11 below, the clinical benefit of masitinib was improved in the subpopulations of Alzheimer's disease patients as defined herein, as compared to the clinical benefit of masitinib observed in the overall study population.

For example, with regards to the clinical marker of ‘time from diagnosis until treatment initiation’, as compared to the masitinib treatment-effect (with respect to placebo) observed in terms of change of ADAS-Cog from baseline in the overall study population, the masitinib treatment-effect in the subpopulation of Alzheimer's disease patients defined as having ‘time from diagnosis to treatment initiation of less than or equal to 3 years’ showed a 34% improvement (see Table 7 below). Similarly, as compared to the masitinib treatment-effect (with respect to placebo) observed in terms of change of ADAS-Cog from baseline in the overall study population, the masitinib treatment-effect in the subpopulation of Alzheimer's disease patients defined as having ‘time from diagnosis to treatment initiation of less than or equal to 2 years’ showed a 29% improvement (see Table 7 below).

TABLE 7
Comparison of masitinib placebo-controlled treatment-effect
(ΔLSM at W24) in subpopulations defined using the clinical
marker of time from diagnosis to
treatment initiation versus overall study population
(according to ADAS-Cog assessment)

| | | | δLSM | ΔLSM | |
|---|---|---|---|---|---|
| | | | (ADAS- | (ADAS- | |
| Population | Arm | N | Cog) | Cog) | Δ_{sub}/Δₒᵥₑᵣₐₗₗ |
| Overall | M4.5 | 182 | −1.4550 | −2.1485 | N/A |
| Population | PBO | 176 | 0.6935 | | |
| Diagnosis ≤2 | M4.5 | 95 | −1.6958 | −2.7754 | 1.29 |
| years | PBO | 96 | 1.0796 | | (29%) |
| Diagnosis ≤3 | M4.5 | 127 | −1.7671 | −2.8700 | 1.34 |
| years | PBO | 133 | 1.1029 | | (34%) |
| Diagnosis ≤4 | M4.5 | 150 | −1.4948 | −2.3435 | 1.09 |
| years | PBO | 155 | 0.8487 | | (9%) |
| Diagnosis ≤5 | M4.5 | 160 | −1.4394 | −2.2599 | 1.05 |
| years | PBO | 161 | 0.8205 | | (5%) |

N: number of patients in subpopulation. ΔLSM: Difference between treatment-arms (treatment-effect) calculated as δLSM[masitinib] minus δLSM[placebo]. δLSM: least-squares means difference of absolute change in ADAS-Cog score at week 24 from baseline. Negative ΔLSM (ADAS-Cog) indicates a greater baseline difference for masitinib as compared with placebo.

For example, with regards to the clinical marker of ‘time from diagnosis until treatment initiation’, as compared to the masitinib treatment-effect (with respect to placebo) observed in terms of change of ADCS-ADL from baseline in the overall study population, the masitinib treatment-effect in the subpopulation of Alzheimer's disease patients defined as having ‘baseline MMSE score greater than or equal to 14 points before treatment initiation’ showed a 32% improvement (see Table 8 below).

TABLE 8
Comparison of masitinib placebo-controlled treatment-effect (ΔLSM at
W24) in subpopulations defined using the clinical marker of baseline MMSE score
versus overall study population (according to ADCS-ADL assessment)

| | | | δLSM | ΔLSM | |
|---|---|---|---|---|---|
| | | | (ADCS- | (ADCS- | |
| Population | Arm | N | ADL) | ADL) | Δ_{sub}/Δₒᵥₑᵣₐₗₗ |
| Overall | M4.5 | 182 | 1.0107 | 1.8190 | N/A |
| Population | PBO | 176 | −0.8083 | | |
| MMSE ≥13 | M4.5 | 179 | 1.1204 | 2.0496 | 1.13 |
| | PBO | 170 | −0.9292 | | (13%) |
| MMSE ≥14 | M4.5 | 164 | 1.3505 | 2.4046 | 1.32 |
| | PBO | 156 | −1.0541 | | (32%) |
| MMSE ≥15 | M4.5 | 151 | 1.3520 | 2.2738 | 1.25 |
| | PBO | 140 | −0.9218 | | (25%) |
| MMSE ≥16 | M4.5 | 140 | 1.6088 | 2.5566 | 1.41 |
| | PBO | 130 | −0.9478 | | (41%) |
| MMSE ≥17 | M4.5 | 128 | 1.0330 | 2.2870 | 1.26 |
| | PBO | 123 | −1.2540 | | (26%) |

N: number of patients in subpopulation. ΔLSM: Difference between treatment-arms (treatment-effect) calculated as δLSM[masitinib] minus δLSM[placebo]. δLSM: least-squares means difference of absolute change in ADCS-ADL score at week 24 from baseline. Positive ΔLSM (ADCS-ADL) indicates a greater baseline difference for masitinib as compared with placebo.

Moreover, as compared to the masitinib treatment-effect (with respect to placebo) observed in terms of change of ADCS-ADL from baseline in the overall study population, the masitinib treatment-effect in the subpopulation of Alzheimer's disease patients defined as having ‘baseline MMSE score ranging from 25 to 21 points before treatment initiation’ showed a 51% improvement (see Table 9 below).

TABLE 9
Comparison of masitinib placebo-controlled treatment-effect (ΔLSM at
W24) in subpopulations defined using the clinical marker of baseline MMSE score
versus overall study population (according to ADCS-ADL assessment)

| | | | δLSM | ΔLSM | |
|---|---|---|---|---|---|
| | | | (ADCS- | (ADCS- | |
| Population | Arm | N | ADL) | ADL) | Δ_{sub}/Δₒᵥₑᵣₐₗₗ |
| Overall | M4.5 | 182 | 1.0107 | 1.8190 | N/A |
| Population | PBO | 176 | −0.8083 | | |
| MMSE [25-21] | M4.5 | 63 | 3.5738 | 2.7435 | 1.51 |
| | PBO | 61 | 0.8302 | | (51%) |
| MMSE [20-12] | M4.5 | 119 | −0.4369 | 1.3770 | 0.76 |
| | PBO | 115 | −1.8139 | | (−24%) |

N: number of patients in subpopulation. ΔLSM: Difference between treatment-arms (treatment-effect) calculated as δLSM[masitinib] minus δLSM[placebo]. δLSM: least-squares means difference of absolute change in ADCS-ADL score at week 24 from baseline. Positive ΔLSM (ADCS-ADL) indicates a greater baseline difference for masitinib as compared with placebo.

Similarly, as compared to the masitinib treatment-effect (with respect to placebo) observed in terms of change of ADAS-Cog from baseline in the overall study population, the masitinib treatment-effect in the subpopulation of Alzheimer's disease patients defined as having ‘baseline MMSE score ranging from 25 to 21 points before treatment initiation’ showed a 35% improvement (see Table 10 below).

TABLE 10
Comparison of masitinib placebo-controlled treatment-effect (ΔLSM at
W24) in in subpopulations defined using the clinical marker of baseline MMSE score
versus overall study population's placebo-controlled treatment effect (according to
ADAS-Cog assessment)

| | | | δLSM | ΔLSM | |
|---|---|---|---|---|---|
| | | | (ADAS- | (ADAS- | |
| Population | Arm | N | Cog) | Cog) | Δ_{sub}/Δₒᵥₑᵣₐₗₗ |
| Overall | M4.5 | 182 | −1.4550 | −2.1485 | N/A |
| Population | PBO | 176 | 0.6935 | | |
| MMSE [25-21] | M4.5 | 63 | −2.4719 | −2.8929 | 1.35 |
| | PBO | 61 | 0.4210 | | (35%) |
| MMSE [20-12] | M4.5 | 119 | −1.0350 | −1.7387 | 0.81 |
| | PBO | 115 | 0.7037 | | (−19%) |

N: number of patients in subpopulation. ΔLSM: Difference between treatment-arms (treatment-effect) calculated as δLSM[masitinib] minus δLSM[placebo]. δLSM: least-squares means difference of absolute change in ADAS-Cog score at week 24 from baseline. Negative ΔLSM (ADAS-Cog) indicates a greater baseline difference for masitinib as compared with placebo.

For example, with regards to the clinical marker of ‘baseline ADCS-ADL score’, as compared to the masitinib treatment-effect (with respect to placebo) observed in terms of change of ADCS-ADL from baseline in the overall study population, the masitinib treatment-effect in the subpopulation of Alzheimer's disease patients defined as having ‘ADCS-ADL score greater than or equal to 50 points before treatment initiation’ showed a 78% improvement (see Table 11 below). Similarly, as compared to the masitinib treatment-effect (with respect to placebo) observed in terms of change of ADCS-ADL from baseline in the overall study population, the masitinib treatment-effect in the subpopulation of Alzheimer's disease patients defined as having ‘ADCS-ADL score greater than 55 points before treatment initiation’ showed a 75% improvement (see Table 11 below).

TABLE 11
Comparison of masitinib placebo-controlled treatment-effect (ΔLSM at
W24) in subpopulations defined using the clinical marker of baseline ADCS-ADL
score versus overall study population (according to ADCS-ADL assessment)

| | | | δLSM | ΔLSM | |
|---|---|---|---|---|---|
| | | | (ADCS- | (ADCS- | |
| Population | Arm | N | ADL) | ADL) | Δ_{sub}/Δₒᵥₑᵣₐₗₗ |
| Overall | M4.5 | 182 | 1.0107 | 1.8190 | N/A |
| Population | PBO | 176 | −0.8083 | | |
| Baseline | M4.5 | 162 | 0.9536 | 1.9799 | 1.09 |
| ADCS-ADL | | | | | |
| ≥32 | PBO | 158 | −1.0263 | | (9%) |
| Baseline | M4.5 | 151 | 0.9909 | 2.0209 | 1.11 |
| ADCS-ADL | | | | | |
| ≥35 | PBO | 149 | −1.0300 | | (11%) |
| Baseline | M4.5 | 146 | 1.0071 | 2.3285 | 1.28 |
| ADCS-ADL | | | | | |
| ≥38 | PBO | 138 | −1.3214 | | (28%) |
| Baseline | M4.5 | 138 | 0.6655 | 2.0733 | 1.14 |
| ADCS-ADL | | | | | |
| >39 | PBO | 134 | −1.4078 | | (14%) |
| Baseline | M4.5 | 134 | 0.8077 | 2.0192 | 1.11 |
| ADCS-ADL | | | | | |
| ≥41 | PBO | 133 | −1.2114 | | (11%) |
| Baseline | M4.5 | 125 | 1.0869 | 2.1676 | 1.19 |
| ADCS-ADL | | | | | |
| >47 | PBO | 117 | −1.0806 | | (19%) |
| Baseline | M4.5 | 93 | 0.7638 | 3.2315 | 1.78 |
| ADCS-ADL | | | | | |
| ≥50 | PBO | 85 | −2.4677 | | (78%) |
| Baseline | M4.5 | 89 | 0.8306 | 3.1871 | 1.75 |
| ADCS-ADL | | | | | |
| >55 | PBO | 81 | −2.3565 | | (75%) |

N: number of patients in subpopulation. ΔLSM: Difference between treatment-arms (treatment-effect) calculated as δLSM[masitinib] minus δLSM[placebo]. δLSM: least-squares means difference of absolute change in ADCS-ADL score at week 24 from baseline. Positive ΔLSM (ADCS-ADL) indicates a greater baseline difference for masitinib as compared with placebo.

1-15. (canceled)

16. A method for treating Alzheimer's disease in a patient in need thereof, comprising administering to the patient masitinib, or a pharmaceutically acceptable salt or solvate thereof, wherein before treatment initiation with masitinib, or the pharmaceutically acceptable salt or solvate thereof, the patient suffering from Alzheimer's disease has an Alzheimer's Disease Cooperative Study—Activities of Daily Living (ADCS-ADL) score equal to or greater than 32, and/or has a Mini-Mental State Examination (MMSE) score equal to or greater than 13, and/or has a time from diagnosis to treatment initiation with masitinib, or the pharmaceutically acceptable salt or solvate thereof, equal to or less than 5 years, and/or has an Alzheimer's Disease Assessment Scale—Cognitive Subscale (ADAS-Cog) score equal to or lower than 40.

17. The method according to claim 16, wherein the patient suffering from Alzheimer's disease has an ADCS-ADL score equal to or greater than 32, 35, 38, 39, 41, 47, 50, or 55, before treatment initiation with masitinib, or the pharmaceutically acceptable salt or solvate thereof.

18. The method according to claim 16, wherein the patient suffering from Alzheimer's disease has an ADCS-ADL score greater than 32, 35, 38, 39, 41, 47, 50, or 55, before treatment initiation with masitinib, or the pharmaceutically acceptable salt or solvate thereof.

19. The method according to claim 16, wherein the patient suffering from Alzheimer's disease has an ADCS-ADL score equal to or greater than 38 or 50 before treatment initiation with masitinib, or the pharmaceutically acceptable salt or solvate thereof.

20. The method according to claim 16, wherein the patient suffering from Alzheimer's disease has an ADCS-ADL score greater than 39 or 55 before treatment initiation with masitinib, or the pharmaceutically acceptable salt or solvate thereof.

21. The method according to claim 16, wherein the patient suffering from Alzheimer's disease has a MMSE score equal to or greater than 13, 14, 15, 16, or 17 before treatment initiation with masitinib, or the pharmaceutically acceptable salt or solvate thereof.

22. The method according to claim 16, wherein the patient suffering from Alzheimer's disease has a MMSE score equal to or greater than 14 before treatment initiation with masitinib, or the pharmaceutically acceptable salt or solvate thereof.

23. The method according to claim 16, wherein the patient suffering from Alzheimer's disease has a MMSE score ranging from 21 to 25 before treatment initiation with masitinib, or the pharmaceutically acceptable salt or solvate thereof.

24. The method according to claim 16, wherein the patient suffering from Alzheimer's disease has a time from diagnosis to treatment initiation with masitinib, or the pharmaceutically acceptable salt or solvate thereof, equal to or less than 5 years, 4 years, 3 years, or 2 years.

25. The method according to claim 16, wherein the patient suffering from Alzheimer's disease has a time from diagnosis to treatment initiation with masitinib, or the pharmaceutically acceptable salt or solvate thereof, equal to or less than 3 years.

26. The method according to claim 16, wherein the patient suffering from Alzheimer's disease has an ADAS-Cog score equal to or lower than 40, 35, 32, or 25 before treatment initiation with masitinib, or the pharmaceutically acceptable salt or solvate thereof.

27. The method according to claim 16, wherein the patient suffering from Alzheimer's disease has an ADAS-Cog score equal to or lower than 35 before treatment initiation with masitinib, or the pharmaceutically acceptable salt or solvate thereof.

28. The method according to claim 16, wherein the pharmaceutically acceptable salt of masitinib is masitinib mesilate.

29. The method according to claim 16, wherein masitinib, or the pharmaceutically acceptable salt or solvate thereof, is administered at a dose ranging from about 1 to about 12 mg/kg/day (mg per kilo body weight per day).

30. The method according to claim 16, wherein masitinib, or the pharmaceutically acceptable salt or solvate thereof, is administered at a dose of about 3 mg/kg/day, about 4.5 mg/kg/day, or about 6 mg/kg/day.

31. The method according to claim 16, wherein masitinib, or the pharmaceutically acceptable salt or solvate thereof, is administered at an initial dose of about 4.5 mg/kg/day during at least 12 weeks, and then at a dose of about 6 mg/kg/day thereafter, with each dose escalation being subjected to toxicity controls.

32. The method according to claim 16, wherein masitinib, or the pharmaceutically acceptable salt or solvate thereof, is administered orally.

33. The method according to claim 16, wherein masitinib, or the pharmaceutically acceptable salt or solvate thereof, is administered with at least one further pharmaceutically active agent.

34. The method according to claim 33, wherein the at least one further pharmaceutically active agent is selected from the group consisting of donepezil, rivastigmine, galantamine, and memantine.
